SEQUENCE LISTING (14 sequences)

SEQ ID NO: 1 - protein, 274 aa, homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 2 - protein, 274 aa, homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEASEGNITVTCRASSFYPRNITLTWRQDGVSLSHDTQQWGDVLPDGNGTYQTWVATRICQGEEQRFTCYMEHSGNHSTHPVPS

SEQ ID NO: 3 - protein, 274 aa, homo sapiens
EPHSLRYNLTVLSWDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 4 - protein, 274 aa, homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESGVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPS

SEQ ID NO: 5 - protein, 274 aa, homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESSVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPS

SEQ ID NO: 6 - protein, 383 aa, homo sapiens [sequence omitted]

SEQ ID NO: 7 - protein, 119 aa, mus musculus
EIQLQQSGPELEKPGASVKISCKASGYAFTGYYMNWMKQRNGKSLDWIGTINPYYGSSTYNQKFKGKATLTVDESSSTAYMQLTSLTSEDSAVYYCARVDGDHGYFDYWGRGTTLTVSS

SEQ ID NO: 8 - protein, 112 aa, mus musculus
DVVMTQTPLSLPVSLGDQASISCRSSQSLVHSNGNTYLHWYLQKPGQSPKLLIYKVSTRFSGVPDRFSGSGSGTDFTLKISRVEAEDLGVYFCSQSTHVPFTFGSGTKLEIK

SEQ ID NO: 9 - protein, 5 aa, mus musculus
GYYMN

SEQ ID NO: 10 - protein, 17 aa, mus musculus
TINPYYGSSTYNQKFKG

SEQ ID NO: 11 - protein, 10 aa, mus musculus
VDGDHGYFDY

SEQ ID NO: 12 - protein, 16 aa, mus musculus
RSSQSLVHSNGNTYLH

SEQ ID NO: 13 - protein, 7 aa, mus musculus
KVSTRFS

SEQ ID NO: 14 - protein, 9 aa, mus musculus
SQSTHVPFT

(19) United States
(12) Patent Application Publication
(10) Pub. No.: US 2023/0296611 A1
(43) Pub. Date: Sep. 21, 2023

(54) CELL SURFACE MICA AND MICB DETECTION USING ANTIBODIES

(71) Applicant: INNATE PHARMA, MARSEILLE (FR)

(72) Inventors: MARION BENEZECH, AURIOL (FR); ROMAIN REMARK, MARSEILLE (FR)

(21) Appl. No.: 18/020,436

(22) PCT Filed: Aug. 6, 2021
(86) PCT No.: PCT/EP2021/072006

§ 371 Date: Feb. 9, 2023

Related U.S. Application Data
(60) Provisional application No. 63/063,475, filed on Aug. 10, 2020.

(51) Int. Cl. G01N 33/574; G01N 33/569; C07K 16/28

(52) U.S. Cl. CPC G01N 33/57492; G01N 33/56977; C07K 16/2833; G01N 2800/52; C07K 2317/31

(57) ABSTRACT

The present disclosure relates to research and diagnostic tools to specifically detect MICA and MICB polypeptides in paraffin embedded tissue samples. The disclosure also relates to methods of using the tools to detect MICA and MICB polypeptides, notably in tumor tissue.

### CROSS-REFERENCE TO RELATED APPLICATIONS

This application claims the benefit of U.S. Provisional Application No. 63/063,475 filed 10 Aug. 2020; which is incorporated herein by reference in its entirety; including any drawings.

### REFERENCE TO SEQUENCE LISTING

The present application is being filed along with a Sequence Listing in electronic format. The Sequence Listing is provided as a file entitled “MICA4_ST25”, created 4 Aug. 2021, which is 19 KB in size. The information in the electronic format of the Sequence Listing is incorporated herein by reference in its entirety.

### FIELD OF THE INVENTION

The present invention relates to research and diagnostic tools to detect proteins of interest in paraffin embedded tissue samples. The invention also relates to methods of using the tools to detect polypeptides, notably in tumor tissue.

### BACKGROUND

MICA (major histocompatibility complex class I-related chain A) and MICB (major histocompatibility complex class I-related chain B) are transmembrane proteins induced by stress signals on infected cells and tumor cells. MICA is a ligand of NKG2D (natural killer group 2, member D) which is an activating receptor expressed on natural killer (NK) cells and on subpopulations of γδ T cells. The interaction between MICA and NKG2D leads to an activation of effector cells which mediate the lysis of target cells. However, the MICA protein undergoes shedding by metalloproteases in the tumor microenvironment and the soluble MICA, in large quantities, correlated with NKG2D down-modulation on peripheral lymphocytes from patients with various cancers. In addition to soluble MICA, membrane-bound MICA is also highly effective in down-modulating NKG2D. These mechanisms allow tumor escape from the control by the immune system.

Many tumor types have been reported to express and/or shed MICA. Tumor have also been reported to express MICB. Antibodies for detection or diagnostic use have generally focused on detection of the soluble (shed) MICA rather than cell bound MICA. However, assessment of cell bound MICA and/or MICB by antibody-based methods may also be valuable. This is because assessment of cell bound MICA and/or MICB is preferably carried out by direct detection of protein as a result of the quantification of MICA/B mRNA not being predictive of the amounts of the corresponding proteins expressed at the cell surface due to the complex processes regulating these transcripts (Raulet et al. 2013 Annu. Rev. Immunol. 2013; 31:413-441).

New methods of treatment of cancer are needed that are able to more specifically make use of the immune system to target cancer cells and, as such, avoid the side effects typical of traditional chemotherapeutic agents. In order to better understand the tumor environment it is often desirable to detect proteins of interest present in tumor tissue and/or at the tumor periphery or otherwise in nearby tissue. This can be done for example using frozen tissue samples. This is not only useful in research but can also help in the decision about what type of treatment to use, for example, by detecting whether a tissue (e.g. a tumor environment) is characterized by presence of a protein that is the target of a treatment (e.g. an immunotherapy). The information can be valuable in order to select a treatment that is capable of modulating the activity of the protein and/or of the cells expressing it.

In addition to frozen tissue, markers can also be detected from tissue samples that have been preserved as formaldehyde (e.g. formalin)-fixed paraffin embedded (FFPE) samples. Following deparaffination, the slides are amenable to, e.g., immunohistochemical methods to detect the expression of specific proteins. The methods have been used routinely to detect tumor antigens in tumor tissue samples. Unfortunately, it is often impossible to find monoclonal antibodies that work effectively and with specificity in FFPE sections. This is believed to be due to the impact of the formalin fixation on structure of proteins. Epitopes bound by antibodies described as being specific on recombinant protein or cells are often present on other proteins when used in FFPE, rendering the antibodies non-specific. In other cases, many epitopes on native cellular protein are destroyed by formalin fixation, causing antibodies identified using recombinant protein or cells to be ineffective for staining FFPE sections. As a result, many receptors have not been amenable to generation of a ligand that binds with specificity in paraffin embedded sections (e.g., no specific epitope remains available following formalin fixation).

Improved diagnostic methods and tools are needed, for example in order to identify the treatments that are most suitable for a given patient.

### SUMMARY OF THE INVENTION

The invention relates, inter alia, to the study, detection and/or monitoring of cell surface MICA and MICB proteins in tissue samples, particularly FFPE tissue samples. The present disclosure arises from the development of methods to detect MICA and MICB proteins on human tumor samples. In particular, through development of a highly specific testing method capable of detecting low levels of MICA*001, MICA*008 and MICB in FFPE tissue samples, including lower levels as may be present when considering solely membranar or cell surface expression. Applicants provide for staining of numerous tumor for MICA and MICB (e.g. a combination of MICA and MICB) in FFPE tissue samples. Staining in FFPE samples, particularly membrane or cell surface staining which requires ability to detect low levels of protein, can be particularly useful to identify tumors suitable for treatment with depleting anti-MICA agents.

The antibodies retain specificity for MICA and MICB polypeptides in FFPE protocols, notably they bind an epitope present on MICA and MICB polypeptides, that remains present and specific following formalin fixation. Yet further, the epitope on the MICA polypeptides remains present and specific following formalin fixation for both of the high predominance MICA alleles in the human population, MICA*001 and *008. The antibodies permitted high specificity of detection of antigen in IHC protocols. Through use of a method of making antibodies that uses paraffin-embedded cell pellets generated from cells bearing at their surface antibody-bound target antigen (cells expressing target antigen pre-incubated with therapeutic antibodies against the target antigen), the inventors obtained antibodies that recognize antigen-specific epitopes in FFPE material while be able to recognize multiple MICA alleles and additionally MICB. The resulting diagnostic antibodies can serve as a universal single-antibody based composition, kit, system or method for consistent detection of target antigen in FFPE samples from patients. The reagents can be used without the need to use additional multiple allele-specific antibodies for the detection of MICA in tissue samples. The reagents can be used to detect MICA and/or MICB expressing tumors having relatively low levels of MICA and/or MICB. Because malignant cells in tumor tissue can express MICA and/or MICB, the reagents can provide increased sensitivity to detect tumor that are positive for at least one of MICA and MICB, and yet further can provide increased ability to identify subjects that can benefit from treatment with a therapeutic that binds both MICA and MICB agent (e.g., an NKG2A protein or fragment, an anti-MICA/B antibody). The reagents can be used to detect MICA and/or MICB at the cell surface or cell membrane of tumor cells (e.g., where levels of MICA and/or MICB are lower as compared to when cytosolic proteins are also included in the assessment). The reagents can be used to select or identify individuals that can then be treated with any suitable anti-MICA antibody, including MICA allele-specific therapeutic antibodies and MICA allele pan-specific therapeutic antibodies that can recognize more than one MICA allele (for example two or more of the most common MICA alleles in the human population, such as MICA*001 and *008).

Formaldehyde-fixed (e.g., formalin-fixed, paraformaldehyde-fixed), paraffin-embedded (FFPE) tissue provide two main advantages over other immunologic methods: (1) the tissue does not require special handling; and (2) cytologic and architectural features are well perceived, allowing for improved histopathologic interpretation. In the examples herein, cells bearing different MICA alleles at the cell surface, cells bearing MICB at the cell surface, and cells bearing MICA or MICB polypeptides at different levels of combined expression were each separately formalin fixed and prepared as paraffin-embedded (FFPE) samples. Use of these samples resulted in the discovery of anti-MICA antibodies for use in staining and study of MICA in human FFPE tissue samples useful as a single reagent across human populations and moreover suitable for detecting lower levels of combined MICA and MICB expression on cells in FFPE samples, e.g. at the surface of tumor cells or more generally cells in tumor tissue. The resulting antibodies were tested in a variety of tumor tissues from human donors and found to retain excellent performance in detection of target antigen in FFPE tissue section. As shown, when compared to comparator antibody which was not consistently able to detect lower levels of combined MICA and MICB expression on cells in FFPE samples, the antibody of the disclosure was able to identify tissue samples as being MICA and/or MICB positive at the cell surface (membranar staining) for tumor types that tested negative for MICA or MICB at the cell surface using the comparator antibody. The antibody therefore has the advantage of permitting detection of a wider range of MICA and/or MICB positive tumors in FFPE samples from individuals, in turn allowing treatment of the individuals having MICA and/or MICB positive tumors with a MICA and/or MICB targeted agent (e.g. an anti-MICA and/or anti-MICB depleting agent).

In one embodiment the disclosure provides a method of producing an antibody that specifically binds to MICA and MICB polypeptides in paraffin-embedded tissues, said method comprising the steps of: a) providing a plurality of candidate antibodies; and b) preparing or selecting antibodies from said plurality that specifically bind to MICA and MICB polypeptides expressed by cells prepared as a paraffin embedded cell sample (e.g. compared to cells prepared as a paraffin embedded cell sample that do not express the MICA or MICB polypeptides).

In one embodiment the disclosure provides a method of producing an antibody that specifically binds to MICA and MICB polypeptides in paraffin-embedded tissues, said method comprising the steps of:
a) providing cells that express MICA polypeptides (e.g., and that do not express MICB polypeptide) at their surface and preparing a paraffin embedded cell sample from such cells;
b) providing cells that express MICB polypeptides (e.g., and that do not express MICA polypeptide) at their surface and preparing a paraffin embedded cell sample from such cells; and
c) providing a plurality of candidate antibodies and preparing or selecting antibodies from said plurality that (i) bind to MICA polypeptides in the paraffin embedded cell sample of step a) and that (ii) bind to MICB polypeptides in the paraffin embedded cell sample of step b), optionally without binding to a paraffin embedded cell sample prepared from cells that lack expression of MICA polypeptides and MICB polypeptides.

In one embodiment the disclosure provides a method of producing an antibody that specifically binds to MICA and MICB polypeptides in paraffin-embedded tissues, said method comprising the steps of:
a) providing cells that express a first allele of MICA (e.g. MICA*001 or MICA*008) polypeptides (e.g., that express said first allele as the sole MICA polypeptide and that do not express MICB polypeptides) at their surface and preparing a paraffin embedded cell sample from such cells;
b) providing cells that express a second allele of MICA that is not the same as the first allele (e.g. the cells express the second allele as the sole MICA polypeptide and that do not express MICB polypeptides)at their surface and preparing a paraffin embedded cell sample from such cells;
c) providing cells that express MICB polypeptides (e.g., and that do not express MICA polypeptide) at their surface and preparing a paraffin embedded cell sample from such cells; and
d) providing a plurality of candidate antibodies and preparing or selecting antibodies from said plurality an antibody that (i) binds to the first MICA allele polypeptide in the paraffin embedded cell sample of step a), (ii) binds to the second MICA allele polypeptide in the paraffin embedded cell sample of step b), and (iii) binds to MICB polypeptides in the paraffin embedded cell sample of step c), optionally without binding to a paraffin embedded cell sample prepared from cells that lack expression of the first and second MICA allele polypeptides as well as MICB polypeptides. Optionally, step (d) comprises: providing a plurality of candidate antibodies and preparing or selecting antibodies from said plurality an antibody that (i) binds to MICA*001 polypeptides in the paraffin embedded cell sample of step a), (ii) binds to MICA*008 polypeptides in the paraffin embedded cell sample of step b), and (iii) binds to MICB polypeptides in the paraffin embedded cell sample of step c), optionally without binding to a paraffin embedded cell sample prepared from cells that lack expression of MICA*001 polypeptides, MICA*001 polypeptides and MICB polypeptides.

In one aspect, provided is an antibody or antibody fragment that specifically binds to MICA and MICB polypeptides in paraffin-embedded tissues obtained by a method of producing an antibody of the disclosure. In one aspect, provided are methods of detecting MICA and/or MICB (e.g. MICA and/or MICB-expressing cells) in a formalin-treated and/or paraffin-embedded tissue sample using the antibodies obtained by a method of producing an antibody of the disclosure.

In one aspect, provided is a method of detecting MICA and/or MICB (e.g. MICA and/or MICB-expressing cells) in a formalin-treated and/or paraffin-embedded tissue sample, optionally a tumor or tumor adjacent tissue sample, optionally a sample from an individual who has received prior treatment with a therapeutic agent (e.g. a chemotherapeutic agent), the method comprising the steps of a) contacting the tissue sample with an anti-MICA/B antibody (e.g. a diagnostic antibody of the disclosure); and b) detecting the presence of the bound antibody in the tissue sample. If MICA and/or MICB is detected, the sample can be determined as comprising MICA/B (e.g. MICA-expressing cells, MICB-expressing tumor cells, MICA- and MICB-expressing cells).

In one embodiment, the disclosures provides a method of detecting MICA/B expressing cells (e.g. cells expressing MICA and/or MICB at their surface or cell membrane) in a sample from a human tumor, the method comprising bringing a paraffin-embedded tumor tissue sample from the individual into contact with an antibody capable of specifically binding to human MICA and MICB polypeptides in a paraffin-embedded tumor tissue sample; and detecting the presence of bound antibody within the section, optionally further detecting cell membrane (cell surface) staining by the antibody.

In any embodiment, the antibody is capable of specifically binding to human MICA and MICB polypeptides in a paraffin-embedded tumor tissue sample can be characterized as being capable of binding and/or staining BxPC-3 cells that have been prepared as a paraffin-embedded cell pellet; optionally wherein the antibody or antibody fragment is capable of binding and/or staining BxPC-3 cells that have been prepared as a paraffin-embedded cell pellet when the antibody is provided at low concentration (1 μg/mL), optionally further wherein the antibody or antibody fragment is capable of binding and/or staining BxPC-3 cells that have been prepared as a paraffin-embedded cell pellet, at each of low concentration (1 μg/mL), at medium (5 μg/mL) and at high concentrations (10 μg/mL) of antibody. Optionally, the antibody or antibody fragment is capable of binding and/or staining the paraffin-embedded BxPC-3 cells consistently, e.g. the binding and/or staining is observed each time when the testing is repeated a plurality of times (e.g. 10 times, 20 times, 100 times or more).

In one embodiment, the antibody capable of specifically binding to human MICA and MICB polypeptides in a paraffin-embedded tumor tissue sample is antibody 12C9, an antibody having the heavy and light chain variable regions thereof, or a function-conservative variant of any of the foregoing.

In one embodiment, the antibody competes with an antibody comprising a heavy chain variable region having the amino acid sequence of SEQ ID NO: 7 and a light chain variable region having the amino acid sequence of SEQ ID NO: 8, for binding to a human MICA and/or MICB polypeptide in a paraffin embedded cell sample (e.g., MICA and/or MICB expressing cells prepared as a paraffin embedded cell sample).

In one aspect, provided is an antibody or antibody fragment capable of specifically binding to a human MICA and/or MICB polypeptide (e.g., a MICA and/or MICB polypeptide in a sample of MICA-expressing cells that have been prepared as a paraffin-embedded cell pellet), wherein the antibody or antibody fragment comprises a heavy chain variable region comprising an amino acid sequence at least 70%, 80% or 90% identical to the amino acid sequence of SEQ ID NO: 7, and a light chain variable region comprising an amino acid sequence at least 70%, 80% or 90% identical to the amino acid sequence of SEQ ID NO: 8.

In one embodiment, provided is an antibody or antibody fragment comprising a heavy chain variable region having the amino acid sequence of SEQ ID NO: 7 and a light chain variable region having the amino acid sequence of SEQ ID NO: 8, or a function-conservative variant thereof.

In one aspect, provided is an antibody or antibody fragment capable of specifically binding to human MICA and/or MICB polypeptides (e.g., a MICA and/or MICB polypeptide in a sample of MICA-expressing cells that have been prepared as a paraffin-embedded cell pellet), wherein the antibody or antibody fragment comprises the three CDRs of the heavy chain variable region sequence of SEQ ID NO: 7 and the three CDRs of the light chain variable region sequence of SEQ ID NO: 8. In one embodiment, the antibody or antibody fragment is conjugated or covalently bound to a detectable moiety. In one embodiment, the antibody or antibody fragment binds to a MICA polypeptide in a sample of MICA-expressing cells that has been prepared as a paraffin-embedded cell pellet, and binds to a MICB polypeptide in a sample of MICB-expressing cells that has been prepared as a paraffin-embedded cell pellet, but does not bind to cells that are MICA-negative and MICB-negative and that have been prepared as a paraffin-embedded cell pellet.

In any embodiment, an antibody or antibody fragment can be characterized as further being capable of binding to a human MICA*004 polypeptide and/or a human MICA*007 polypeptide, for example the antibody or antibody fragment binds to MICA*004 and/or MICA*007 polypeptides in a paraffin embedded cell sample.

In any embodiment, detecting MICA/B expressing cells in a comprise the steps of:
obtaining a biological sample (e.g. as a biopsy, as a cell pellet) comprising cells (e.g., tumor cells, MICA and/or MICB-expressing cells);
fixing, embedding in paraffin, sectioning, and deparaffinizing the sample, and optionally transferring the sample to a slide;
bringing said section into contact with an antibody that specifically binds to a human MICA and MICB polypeptide; and
detecting the presence of bound antibody within the section.

In other embodiments, provided are methods for making or producing antibodies. In other embodiments, provided are kits comprising monoclonal antibodies that bind an antigen in a paraffin embedded cell sample having the features disclosed herein (diagnostic antibodies), and a second antibody (e.g. a therapeutic antibody) capable of specifically binding to the same target antigen as the diagnostic antibody. In certain embodiments, provided is use of monoclonal antibodies having the features disclosed herein in immunohistochemistry assays (including but not limited to antibodies and antibody fragments that bind a human MICA and MICB polypeptide), in diagnostic methods (including but not limited to use in companion diagnostics, e.g. to select individuals for treatment with a therapeutic antibody), in prognostic methods, in patient monitoring methods.

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, more optionally by “consisting of”.

As used herein, “paraffin-embedded sample” (or paraffin-embedded “cells”, “cell pellet”, “slides”, or “tissues”) refers to cells or tissues taken from an organism or from in vitro cell culture that have been fixed, embedded in paraffin, sectioned, deparaffinized, and transferred to a slide. It will be appreciated that fixation and paraffin embedding is a common practice that can vary in many aspects, e.g., with respect to the fixation and embedding methods used, with respect to the protocol followed, etc., and that for the purposes of the present invention any such variant method is encompassed, so long as it involves fixation of the tissue (such as by formalin treatment), embedding in paraffin or equivalent material, sectioning and transfer to a slide.

The term “biological sample” or “sample” as used herein includes but is not limited to a biological fluid (for example serum, lymph, blood), cell sample, or tissue sample (for example bone marrow or tissue biopsy such as skin, breast, lung, colon, ovary, stomach, mucosal tissue such as from the gut, gut lamina propria).

The term “antibody” herein is used in the broadest sense and specifically includes full-length monoclonal antibodies, polyclonal antibodies, multispecific antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988). An “antibody fragment” comprises a portion of a full-length antibody, e.g. antigen-binding or variable regions thereof. Examples of antibody fragments include Fab, Fab′, F(ab)₂, F(ab′)₂, F(ab)₃, Fv (typically the VL and VH domains of a single arm of an antibody), single-chain Fv (scFv), dsFv, Fd fragments (typically the VH and CH1 domain), and dAb (typically a VH domain) fragments; VH, VL, VhH, and V-NAR domains; minibodies, diabodies, triabodies, tetrabodies, and kappa bodies (see, e.g., III et al., Protein Eng 1997; 10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. The application of either definition to refer to a CDR of an antibody or variants thereof is intended to be within the scope of the terms as defined and used herein. The appropriate amino acid residues which encompass the CDRs as defined by commonly used numbering schemes are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1

| CDR | Kabat | Chotia | AbM |
|---|---|---|---|
| HCDR1 | 31-35 | 26-32 | 26-35 |
| HCDR2 | 50-65 | 52-58 | 50-58 |
| HCDR3 | 95-102 | 95-102 | 95-102 |
| VCDR1 | 24-34 | 26-32 | 24-34 |
| VCDR2 | 60-56 | 50-52 | 50-56 |
| VCDR3 | 89-97 | 91-96 | 89-97 |

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes. By “constant light chain” or “light chain constant region” as used herein is meant the region of an antibody encoded by the kappa (Ckappa) or lambda (Clambda) light chains. The constant light chain typically comprises a single domain, and as defined herein refers to positions 108-214 of Ckappa, or Clambda, wherein numbering is according to the EU index (Kabat et al., 1991, Sequences of Proteins of Immunological Interest, 5th Ed., United States Public Health Service, National Institutes of Health, Bethesda). By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant the polypeptide that comprises the VH, CH1, VL, and CL immunoglobulin domains. Fab may refer to this region in isolation, or this region in the context of a polypeptide, multispecific polypeptide or ABD, or any other embodiments as outlined herein.

By “single-chain Fv” or “scFv” as used herein are meant antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994).

By “Fv” or “Fv fragment” or “Fv region” as used herein is meant a polypeptide that comprises the VL and VH domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides include but is not limited to antibodies, Fc fusions and Fc fragments.

By “variable region” as used herein is meant the region of an antibody that comprises one or more Ig domains substantially encoded by any of the VL (including Vkappa (VK) and Vlambda) and/or VH genes that make up the light chain (including kappa and lambda) and heavy chain immunoglobulin genetic loci respectively. A light or heavy chain variable region (VL or VH) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

The term “depleting”, with respect to MICA and/or MICB-expressing cells means a process, method or agent that can kill, eliminate, lyse or induce such killing, elimination or lysis, so as to negatively affect the number of MICA and/or MICB -expressing cells present in a sample or in a subject. An agent can for example comprise an antibody that binds MICA and/or MICB and that directs ADCC (antibody-dependent cellular cytotoxicity) towards MICA and/or MICB-expressing cells, or the agent can be an antibody drug conjugate that binds MICA and that directly causes the death of MICA and/or MICB -expressing cells by delivery of its cytotoxic agent to the cells.

The terms “immunoconjugate” and “antibody conjugate” are used interchangeably and refer to an antigen binding agent, e.g., an antibody binding protein or an antibody that is conjugated to another moiety (e.g., a cytotoxic agent). An immunoconjugate comprising an antigen binding agent conjugated to a cytotoxic agent can also be referred to as a “antibody drug conjugate” or an “ADC”.

The term “agent” is used herein to denote a chemical compound, a mixture of chemical compounds, a biological macromolecule, or an extract made from biological materials. The term “therapeutic agent” refers to an agent that has biological activity.

The term “specifically binds to” means that an antibody or polypeptide can bind preferably in a competitive binding assay to the binding partner, e.g. MICA and MICB, as assessed using either recombinant forms of the proteins, epitopes therein, or native proteins present on the surface of isolated target cells. Competitive binding assays and other methods for determining specific binding are further described below and are well known in the art.

When an antibody or polypeptide is said to “compete with” a particular monoclonal antibody, it means that the antibody or polypeptide competes with the monoclonal antibody in a binding assay using appropriate target molecules or surface expressed target molecules, for example MICA expressed by cells in paraffin-embedded cell pellets. For example, if a test antibody reduces the binding of 12C9 to a MICA polypeptide or MICA-expressing cell in a binding assay, the antibody is said to “compete with 12C9.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein (e.g. heavy or light chains or variable regions thereof) to which it is compared.

The term “affinity”, as used herein, means the strength of the binding of an antibody or polypeptide to an epitope. The affinity of an antibody is given by the dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BlAcore™ SPR analytical device).

The term “epitope” refers to an antigenic determinant, and is the area or region on an antigen to which an antibody or polypeptide binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y05W refers to a variant of a parent polypeptide, in which the tyrosine at position 50 is replaced with tryptophan. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

In the context herein, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

Whenever within this whole specification “treatment of cancer” or the like is mentioned with reference to an anti-MICA binding agent (e.g. an anti-MICA/MICB antibody), there is meant: (a) method of treatment of cancer, said method comprising the step of administering (for at least one treatment) an anti-MICA binding agent, (preferably in a pharmaceutically acceptable carrier material) to an individual, a mammal, especially a human, in need of such treatment, in a dose that allows for the treatment of cancer, (a therapeutically effective amount), preferably in a dose (amount) as specified herein; (b) the use of an anti-MICA binding agent for the treatment of cancer, or an anti-MICA binding agent, for use in said treatment (especially in a human); (c) the use of an anti-MICA binding agent for the manufacture of a pharmaceutical preparation for the treatment of cancer, a method of using an anti-MICA binding agent for the manufacture of a pharmaceutical preparation for the treatment of cancer, comprising admixing an anti-MICA binding agent with a pharmaceutically acceptable carrier, or a pharmaceutical preparation comprising an effective dose of an anti-MICA binding agent that is appropriate for the treatment of cancer; or (d) any combination of a), b), and c), in accordance with the subject matter allowable for patenting in a country where this application is filed.

### Antibodies for Detecting MICA and MICB

MICA (PERB11.1) refers to MHC class I polypeptide-related sequence A (see, e.g.,

UniProtKB/Swiss-Prot Q29983), its gene and cDNA and its gene product, or naturally occurring variants thereof. Nomenclature of MICA genes and proteins, together with reference to accession number of sequence for different alleles are described in Frigoul A. and Lefranc, M-P. Recent Res. Devel. Human Genet., 3(2005): 95-145 ISBN: 81-7736-244-5, the disclosure of which is incorporated herein by reference. MICA genes and protein sequence, including polymorphisms at the protein and DNA level, are also available from http://www.ebi.ac.uk/ipd/imgt/hla/align.html maintained by Cancer Research UK and the European Bioinformatics Institute (EBI).

The amino acid sequences of MICA were first described in Bahram et al (1994) Proc. Nat. Acad. Sci. 91: 6259-6263 and Bahram et al. (1996) Immunogenetics 44:80-81, the disclosures of which are incorporated herein by reference. The MICA gene is polymorphic, displaying an unusual distribution of a number of variant amino acids in their extracellular α1, α2, and α3 domains. To further define the polymorphism of MICA, Petersdorf et al. (1999) examined its alleles among 275 individuals with common and rare HLA genotypes. The amino acid sequence of the extracellular α1, α2, and α3 domains of human MICA are shown in SEQ ID NOS: 1-5. The full MICA sequence further comprises a leader sequence of 23 amino acids, as well as a transmembrane domain and a cytoplasmic domain. The amino acid sequence of MICA*001 is shown in SEQ ID NO: 1, corresponding to Genbank accession no. AAB41060. The amino acid sequence of human MICA allele MICA*004 is shown in SEQ ID NO: 2, corresponding to Genbank accession no. AAB41063. The amino acid sequence of human MICA allele MICA*007 is shown in SEQ ID NO: 3, corresponding to Genbank accession no. AAB41066. The amino acid sequence of human MICA allele MICA*008 is shown in SEQ ID NO: 4, corresponding to Genbank accession no. AAB41067. The amino acid sequence of human MICA allele MICA*019 is shown in SEQ ID NO: 5, corresponding to Genbank accession no. AAD27008.

MICB (also known as PERB11.2) refers to MHC class I polypeptide-related sequence B (See, e.g., UniProtKB/Swiss-Prot Q29980). The amino acid sequence of an exemplary human MICB polypeptide is shown Genbank accession no. CAI18747 (SEQ ID NO: 6).

| MICA | | |
|---|---|---|
| Allele | SEQ ID | Amino acid sequence |
| MICA*001 | 1 | EPHSLRYNLT VLSWDGSVQS GFLTEVHLDG QPFLRCDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETKEWTMP QSSRAQTLAM NVRNFLKEDA |
| | | MKTKTHYHAM HADCLQELRR YLKSGVVLRR TVPPMVNVTR SEASEGNITV |
| | | TCRASGFYPW NITLSWRQDG VSLSHDTQQW GDVLPDGNGT YQTWVATRIC |
| | | QGEEQRFTCY MEHSGNHSTH PVPS |
| MICA*004 | 2 | EPHSLRYNLT VLSWDGSVQS GFLAEVHLDG QPFLRYDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NVETEEWTVP QSSRAQTLAM NVRNFLKEDA |
| | | MKTKTHYHAM HADCLQELRR YLESSVVLRR RVPPMVNVTR SEASEGNITV |
| | | TCRASSFYPR NITLTWRQDG VSLSHDTQQW GDVLPDGNGT YQTWVATRIC |
| | | QGEEQRFTCY MEHSGNHSTH PVPS |
| MICA*007 | 3 | EPHSLRYNLT VLSWDGSVQS GFLAEVHLDG QPFLRCDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTMP QSSRAQTLAM NVRNFLKEDA |
| | | MKTKTHYHAM HADCLQELRR YLKSGVVLRR TVPPMVNVTR SEASEGNITV |
| | | TCRASGFYPW NITLSWRODG VSLSHDTQQW GDVLPDGNGT YQTWVATRIC |
| | | QGEEQRFTCY MEHSGNHSTH PVPS |
| MICA*008 | 4 | EPHSLRYNLT VLSWDGSVQS GFLAEVHLDG QPFLRYDROK CRAKPOGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTVP QSSRAQTLAM NVRNFLKEDA |
| | | MKTKTHYHAM HADCLQELRR YLESGVVLRR TVPPMVNVTR SEASEGNITV |
| | | TCRASSFYPR NIILTWRQDG VSLSHDTQQW GDVLPDGNGT YQTWVATRIC |
| | | RGEEQRFTCY MEHSGNHSTH PVPS |
| MICA*019 | 5 | EPHSLRYNLT VLSWDGSVQS GFLAEVHLDG QPFLRYDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTVP QSSRAQTLAM NVRNFLKEDA |
| | | MKTKTHYHAM HADCLQELRR YLESSVVLRR TVPPMVNVTR SEASEGNITV |
| | | TCRASSFYPR NIILTWRQDG VSLSHDTQQW GDVLPDGNGT YQTWVATRIC |
| | | RGEEQRFTCY MEHSGNHSTH PVPS |
| MICB | 6 | MGLGRVLLFL AVAFPFAPPA AAAEPHSLRY NLMVLSQDGS VQSGFLAEGH |
| | | LDGQPFLRYD ROKRRAKPQG QWAEDVLGAK TWDTETEDLT ENGQDLRRTL |
| | | THIKDQKGGL HSLQEIRVCE IHEDSSTRGS RHFYYDGELF LSQNLETQES |
| | | TVPQSSRAQT LAMNVTNFWK EDAMKTKTHY RAMQADCLOK LORYLKSGVA |
| | | IRRTVPPMVN VTCSEVSEGN ITVTCRASSF YPRNITLTWR QDGVSLSHNT |
| | | QQWGDVLPDG NGTYQTWVAT RIRQGEEQRF TCYMEHSGNH GTHPVPSGKA |
| | | LVLQSQRTDF PYVSAAMPCF VIIIILCVPC CKKKTSAAEG PELVSLQVLD |
| | | QHPVGTGDHR DAAQLGFQPL MSATGSTGST EGA |

While MICA is expressed constitutively in certain cells, low levels of MICA expression do not usually give rise to host immune cell attack. However, on MICA is upregulated on rapidly proliferating cells such as tumor cells. MICA is the most highly expressed of all NKG2D ligands, and it has been found across a wide range of tumor types (e.g., carcinomas in general, bladder cancer, melanoma, lung cancer, hepatocellular cancer, glioblastoma, prostate cancer, hematological malignancies in general, acute myeloid leukemia, acute lymphatic leukemia, chronic myeloid leukemia and chronic lymphatic leukemia. Recently, Tsuboi et al. (2011) (EMBO J: 1-13) reported that the O-glycan branching enzyme, core2 β-1,6-N-acetylglucosaminyltransferase (C2GnT) is active in MICA-expressing tumor cells and that MICA from tumor cells contains core2O-glycan (an O-glycan comprising an N-acetylglucosamine branch connected to N-acetylgalactosamine).

Bauer et al Science 285: 727-729, 1999 provided a role for MICA as a stress-inducible ligand for NKG2D. As used herein, “MICA” refers to any MICA polypeptide, including any variant, derivative, or isoform of the MICA gene or encoded protein(s) to which they refer. The MICA gene is polymorphic, displaying an unusual distribution of a number of variant amino acids in their extracellular alpha-1, alpha-2, and alpha-3 domains. Various allelic variants have been reported for MICA polypeptides (e.g., MICA), each of these are encompassed by the respective terms, including, e.g., human MICA polypeptides MICA*001, MICA*002, MICA*004, MICA*005, MICA*006, MICA*007, MICA*008, MICA*009, MICA*010, MICA*011, MICA*012, MICA*013, MICA*014, MICA*015, MICA*016, MICA*017, MICA*018, MICA*019, MICA*020, MICA*022, MICA*023, MICA*024, MICA*025, MICA*026, MICA*027, MICA*028, MICA*029, MICA*030, MICA*031, MICA*032, MICA*033, MICA*034, MICA*035, MICA*036, MICA*037, MICA*038, MICA*039, MICA*040, MICA*041, MICA*042, MICA*043, MICA*044, MICA*045, MICA*046, MICA*047, MICA*048, MICA*049, MICA*050, MICA*051, MICA*052, MICA*053, MICA*054, MICA*055, MICA*056 and further MICA alleles MICA*057-MICA*087.

As used herein, “NKG2D” and, unless otherwise stated or contradicted by context, the terms “hNKG2D,” “NKG2-D,” “CD314,” “D12S2489E,” “KLRK1,” “killer cell lectin-like receptor subfamily K, member 1,” or “KLRK1,” refer to a human killer cell activating receptor gene, its cDNA (e.g., GenBank Accession No. NM_007360), and its gene product (GenBank Accession No. NP_031386), or naturally occurring variants thereof. In NK and T cells, hNKG2D can form heterodimers or higher order complexes with proteins such as DAP10 (GenBank Accession No. AAG29425, AAD50293). Any activity attributed herein to hNKG2D, e.g., cell activation, antibody recognition, etc., can also be attributed to hNKG2D in the form of a heterodimer such as hNKG2D-DAP10, or higher order complexes with these two (and/or other) components.

The 3D structure of MICA in complex with NKG2D has been determined (see, e.g., Li et al., Nat. Immunol. 2001; 2:443-451; code 1hyr, and in IMGT/3Dstructure-DB (Kaas et al. Nucl. Acids Res. 2004; 32:D208-D210)). When MICA is in complex with a NKG2D homodimer, the residues 63 to 73 (IGMT numbering) of MICA α2 are ordered, adding almost two turns of helix. The two monomers of NKG2D equally contribute to interactions with MICA, and seven positions in each NKG2D monomer interact with one of the MICA α1 or α2 helix domains.

The antibodies (e.g. diagnostic antibodies) of the disclosure bind to human MICA (including MICA*001 and MICA*008, optionally further MICA*004, MICA*007 and/or MICA*019) and MICB with specificity, particularly in fixed samples such as paraffin-embedded tissue sections. The antibodies can specifically bind to their target antigen in a biological sample (e.g. a FFPE section) comprising MICA and MICB expressing cells that have been prepared as a paraffin-embedded cell pellet.

The antibodies can optionally be characterized as an antibody or antibody fragment that binds to a human MICA*001 polypeptide in a sample of MICA*001-expressing cells that has been prepared as a paraffin-embedded cell pellet, that binds to a human MICA*008 polypeptide in a sample of MICA*008-expressing cells that has been prepared as a paraffin-embedded cell pellet, and that binds to a human MICB polypeptide in a sample of MICB-expressing cells that has been prepared as a paraffin-embedded cell pellet, but does not bind to MICB-negative cells (for example Raji cells), that have been prepared as a paraffin-embedded cell pellet. In any embodiment the antibody or antibody fragment can optionally further be characterized as an antibody or antibody fragment that binds to a human MICA*002 polypeptide in a sample of MICA*002-expressing cells that has been prepared as a paraffin-embedded cell pellet. In any embodiment the antibody or antibody fragment can optionally further be characterized as an antibody or antibody fragment that binds to a human MICA*007 polypeptide in a sample of MICA*007-expressing cells that has been prepared as a paraffin-embedded cell pellet. In any embodiment the antibody or antibody fragment can optionally further be characterized as an antibody or antibody fragment that binds to a human MICA*004 polypeptide in a sample of MICA*004-expressing cells that has been prepared as a paraffin-embedded cell pellet.

The ability of the antibodies to specifically bind MICA and MICB in paraffin-embedded tissue sections makes them useful for numerous applications, in particular for detecting MICA- and/or MICB-expressing cells (e.g. tumor cells, cells contributing to tumor progression, cells contributing to escape of tumor from control or lysis by the host immune system) and levels or distribution of MICA- and/or MICB-expressing cells for diagnostic or therapeutic purposes, as described herein. In certain embodiments, the antibodies are used to determine the presence or level of MICA and/or MICB-expressing cells in or near tumor tissue in a sample (e.g. biopsy) taken from an individual, for example an individual having a cancer or tumor. Optionally further, in one embodiment, presence of MICA and/or MICB at the cell (e.g., tumor cell) membrane is assessed and/or detected in the tissue sample. Optionally further, in one embodiment, if MICA and/or MICB is detected in the tissue sample, MICA and/or MICB-expressing cells are determined to be present. Optionally, in another embodiment, if MICA and/or MICB is detected (optionally if a predetermined level of MICA and/or MICB staining is detected) in the tissue sample, the individual is determined to be suited for treatment with a therapeutic antibody that binds MICA and/or MICB, e.g., a depleting anti-MICA and/or MICB antibody. Optionally, in one embodiment, the individual has been treated (e.g. in an ongoing or prior course of therapy) with a therapeutic antibody that binds the target antigen.

The detection of the binding of the antibody to MICA and/or MICB can be performed in any of a number of ways. For example, the antibody can be directly labeled with a detectable moiety, e.g., a luminescent compound such as a fluorescent moiety, or with a radioactive compound, with gold, with biotin (which allows subsequent, amplified binding to avidin, e.g., avidin-AP), or with an enzyme such as alkaline phosphatase (AP) or horseradish peroxidase (HRP). Alternatively, the binding of the antibody to the target antigen in the sample is assessed indirectly, for example by using a secondary antibody that binds to the primary anti-target antigen antibody and that itself is labeled, preferably with an enzyme such as horseradish peroxidase (HRP) or alkaline phosphatase (AP); however, it will be appreciated that the secondary antibodies can be labeled or detected using any suitable method. In a preferred embodiment, an amplification system is used to enhance the signal provided by the secondary antibody, for example the EnVision system in which the secondary antibodies are bound to a polymer (e.g., dextran) that is bound to many copies of a detectable compound or enzyme such as HRP or AP (see, e.g., Wiedorn et al. (2001) The Journal of Histochemistry & Cytochemistry, Volume 49(9): 1067-1071; Kämmerer et al., (2001) Journal of Histochemistry and Cytochemistry, Vol. 49, 623-630; the entire disclosures of which are herein incorporated by reference).

In one aspect the disclosure provides methods producing antibodies that can detect MICA and/or MICB in FFPE samples. MICA and/or MICB or one or more immunogenic fragments thereof can be used as immunogens to raise antibodies, and the antibodies can recognize epitopes within the target antigen polypeptide on paraffin-embedded samples as described herein. Preferably, the recognized epitopes are present on the cell surface, i.e. they are accessible to antibodies present outside of the cell. In one aspect, the epitope is the epitope specifically recognized in a paraffin-embedded cell pellet sample by antibody 12C9. In one aspect, an antibody competes for binding with antibody 12C9 to the epitope specifically recognized by antibody 12C9 in a paraffin-embedded cell pellet sample. Further, antibodies recognizing the same epitope or competing for binding with 12C9 to the epitopes recognized by antibody 12C9 within MICA can be used to bind MICA and/or MICB with maximum efficacy and breadth in a population of human individuals who have or may have received different prior therapies, notably who have or may have been treated with therapeutic antibodies to the target antigen polypeptide (e.g., MICA and/or MICB, MICA*001 and MICA*008 and further MICB).

The antibodies may be produced by a variety of techniques known in the art, for example as set forth in Example 2 herein. Typically, they are produced by immunization of a non-human animal, preferably a mouse, with an immunogen comprising MICA and/or MICB polypeptides (e.g. MICA*001, MICA*008 and MICB polypeptides), preferably human polypeptides. The polypeptide may comprise the full-length sequence of the human polypeptide, or a fragment or derivative thereof, typically an immunogenic fragment, i.e., a portion of the polypeptide comprising an epitope exposed on the surface of cells expressing a MICA polypeptide, preferably the epitope recognized by the 12C9 antibody. Such fragments typically contain at least about 7 consecutive amino acids of the mature polypeptide sequence, even more preferably at least about 10 consecutive amino acids thereof. Fragments typically are essentially derived from the extra-cellular domain of the receptor. In one embodiment, the immunogen comprises a wild-type human MICA polypeptide or fragment thereof. In a specific embodiment, the immunogen comprises intact cells, particularly intact human cells, optionally treated or lysed.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, NY (1988)). The immunogen is suspended or dissolved in a buffer, optionally with an adjuvant, such as complete or incomplete Freund's adjuvant. Methods for determining the amount of immunogen, types of buffers and amounts of adjuvant are well known to those of skill in the art and are not limiting in any way on the present invention. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with an adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be used as long as they result in the production of B cells expressing an antibody directed to the antigen used in immunization.

In an alternate embodiment, lymphocytes from a non-immunized non-human mammal are isolated, grown in vitro, and then exposed to the immunogen in cell culture. The lymphocytes are then harvested and the fusion step described below is carried out.

Splenocytes can be isolated from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the lymphocytes can be fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, U.S.A., X63 Ag8653 and SP-2 cells available from the American Type Culture Collection, Rockville, Maryland U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in Goding, “Monoclonal Antibodies: Principles and Practice,” pp. 59-103 (Academic Press, 1986), the disclosure of which is herein incorporated by reference.

The cells are allowed to grow in the selection media for sufficient time for colony formation and antibody production. This is usually between about 7 and about 14 days.

The hybridoma colonies can be assayed for the production of antibodies that specifically bind to MICA and/or MICB on fixed and paraffin-embedded MICA and/or MICB expressing cell pellet, optionally hybridomas are assayed for competing with antibody 12C9 in binding to the fixed paraffin-embedded MICA and/or MICB-expressing cell pellet. The wells positive for the desired antibody production are examined to determine if one or more distinct colonies are present. If more than one colony is present, the cells may be re-cloned and grown to ensure that only a single cell has given rise to the colony producing the desired antibody. Cells that do not naturally express target antigen (e.g. MICA) can be made to express target antigen (e.g. by transfection with nucleic acids expressing target antigen), prepared as a cell pellet, formalin fixed, embedded in paraffin, sectioned, deparaffinized, and transferred to a slide. Control cells not expressing target antigen (e.g. the same cells as above but not transfected with target antigen) can be used as negative control.

Hybridomas that are confirmed to produce a desired monoclonal antibody can be grown up in larger amounts in an appropriate medium, such as DMEM or RPMI-1640. Alternatively, the hybridoma cells can be grown in vivo as ascites tumors in an animal. After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

Positive wells with a single apparent colony are typically re-cloned and re-assayed to insure only one monoclonal antibody is being detected and produced.

Antibodies may also be produced by selection of combinatorial libraries of immunoglobulins, as disclosed for instance in (Ward et al. Nature, 341 (1989) p. 544, the entire disclosure of which is herein incorporated by reference). For example, libraries may be generated using phage display techniques.

According to an alternate embodiment, hybridomas can first be assayed for the production of antibodies that specifically bind to target antigen polypeptide (e.g. MICA and/or MICB), and the DNA encoding an antibody is isolated from a hybridoma and placed in an appropriate expression vector for transfection into an appropriate host cell. The host cell is then used for the recombinant production of the antibody, or variants thereof, such as a functional fragment of the antibody, chimeric antibodies comprising the antigen recognition portion of the antibody, or versions comprising a detectable moiety. The recombinantly produced antibody can then be assayed for the production of antibodies that specifically bind to MICA and/or MICB on fixed and paraffin-embedded MICA and/or MICB expressing cell pellet, optionally hybridomas are assayed for competing with antibody 12C9 in binding to the fixed paraffin-embedded MICA and/or MICB-expressing cell pellet.

The identification of one or more antibodies that bind(s) to MICA and/or MICB, particularly that compete for binding to MICA and/or MICB (or the epitope thereon bound by 12C9) with monoclonal antibody 12C9, can be readily determined using any one of a variety of immunological screening assays in which antibody competition can be assessed according to the methods described herein, or any other suitable method.

In certain embodiments, one pre-mixes the control antibodies (12C9, for example) with varying amounts of the test antibodies (e.g., about 1:10 or about 1:100) for a period of time prior to applying to the MICA and/or MICB antigen sample (e.g. a paraffin-embedded MICA and/or MICB expressing cell pellet sample). In other embodiments, the control and varying amounts of test antibodies can simply be admixed during exposure to the MICA and/or MICB antigen sample. As long as one can distinguish bound from free antibodies (e. g., by using separation or washing techniques to eliminate unbound antibodies) and 12C9 from the test antibodies (e. g., by using species-specific or isotype-specific secondary antibodies or by specifically labeling 12C9 with a detectable label) one can determine if the test antibodies reduce the binding of 12C9 to the antigens, indicating that the test antibody competes for binding to antigen with 12C9. The binding of the (labeled) control antibodies in the absence of a completely irrelevant antibody can serve as the control high value. The control low value can be obtained by incubating the labeled (12C9) antibodies with unlabelled antibodies of exactly the same type (12C9), where competition would occur and reduce binding of the labeled antibodies. In a test assay, a significant reduction in labeled antibody reactivity in the presence of a test antibody is indicative of a test antibody that “cross-reacts” with the labeled (12C9) antibody. Any test antibody that reduces the binding of 12C9 to MICA and/or MICB by at least about 50%, such as at least about 60%, or more preferably at least about 70% (e. g., about 65-100%), at any ratio of 12C9:test antibody between about 1:10 and about 1:100 is considered to be an antibody that competes with 12C9. Preferably, such test antibody will reduce the binding of 12C9 to the MICA and/or MICB antigen by at least about 90% (e.g., about 95%).

Upon immunization and production of antibodies in a vertebrate or cell (or, e.g., the generation of a library of candidate antibodies, optionally a library of nucleic acid or amino acid sequences for the antibodies), particular selection steps may be performed to isolate antibodies as claimed. In this regard, in a specific embodiment, the invention also relates to methods of producing such antibodies, comprising: (a) providing a library of antibodies and/or immunizing a non-human mammal with an immunogen comprising a MICA and/or MICB polypeptide and preparing antibodies from said immunized animal; and (b) selecting antibodies from step (a) that are capable of specifically binding said MICA and/or MICB polypeptide in a paraffin-embedded cell pellet, e.g, an FFPE cell pellet.

DNA encoding the monoclonal antibodies of the invention, e.g., antibody 12C9, can be readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody.

The disclosure provides a screening method based on formaldehyde-treated paraffin-embedded cell pellets (FFPE cell pellets) that reveal MICA and/or MICB epitopes present following formalin treatment, moreover including when cells are incubated with other anti-MICA antibodies (e.g. neutralizing antibodies) prior to fixation. Accordingly, in one aspect, the disclosure provides a monoclonal antibody that specifically binds MICA and/or MICB polypeptide-expressing cells (e.g. cells made to express MICA and/or MICB) in a sample preserved as a paraffin-embedded cell pellet (and deparaffinized prior to analysis). Optionally the antibody is further characterized by not binding to MICA- and MICB-negative cells (cells that do not express either MICA or MICB) in a paraffin-embedded cell pellet. Optionally the antibody is further characterized by binding to MICA and/or MICB polypeptide-expressing cells in paraffin-embedded tissue sections.

In one aspect the disclosure provides a monoclonal antibody (e.g. a first antibody) that specifically binds human MICA and MICB polypeptides, wherein said antibody (e.g. a first antibody) specifically binds to said MICA and MICB polypeptides in a biological sample that has been treated (or fixed) using formaldehyde (e.g. formalin, paraformaldehyde). Formaldehyde fixation can be used in particular the preparation of paraffin embedded tissue sections which can then be deparaffinized and analyzed for presence of a marker of interest, e.g. MICA.

In one aspect, provided is a monoclonal antibody that specifically binds a human MICA polypeptide expressed by a cell that has been preserved in paraffin, e.g. a cell that has been preserved as a paraffin-embedded cell pellet. Optionally, the cells are pelleted, formaldehyde treated (e.g. formaldehyde, formalin, paraformaldehyde) and then paraffin embedded. Optionally, the cell that expresses the human MICA polypeptide is in a biological sample that has been deparaffinized prior to antibody binding and analysis.

In one aspect, the antibodies bind an antigenic determinant present on MICA and MICB, optionally on MICA*001, MICA*008 and MICB, in a FFPE cell pellet sample. In one aspect, the antibodies bind substantially the same epitope or determinant as antibody 12C9, or compete with 12C9 for binding to such epitope on MICA and/or MICB in a FFPE cell pellet sample. In one embodiment, the antibodies bind to an epitope of MICA and/or MICB that at least partially overlaps with, or includes at least one residue in, the epitope bound by antibody 12C9. The residues bound by the antibody can be specified as being present on the surface of the MICA and MICB polypeptide, optionally further on the surface of a MICA and MICB polypeptide expressed on the surface of a cell, optionally further on the surface of a MICA and MICB polypeptide expressed on the surface of a cell that has been preserved as a paraffin-embedded cell pellet.

The amino acid sequence of the heavy chain variable region of antibody 12C9 is listed as SEQ ID NO: 7, the amino acid sequence of the light chain variable region is listed as SEQ ID NO: 8. In a specific embodiment, an antibody binds essentially the same epitope or determinant as monoclonal antibody 12C9; optionally the antibody comprises the hypervariable region of antibody 12C9. In any of the embodiments herein, antibody 12C9 can be characterized by the amino acid sequences and/or nucleic acid sequences encoding it. In one embodiment, the monoclonal antibody comprises the Fab or F(ab′)₂ portion of 12C9. Also provided is a monoclonal antibody that comprises the heavy chain variable region of 12C9 or a function-conservative variant thereof. According to one embodiment, the monoclonal antibody comprises the three CDRs of the heavy chain variable region of 12C9 Also provided is a monoclonal antibody that further comprises the variable light chain variable region of 12C9 or a function-conservative variant thereof. According to one embodiment, the monoclonal antibody comprises the three CDRs of the light chain variable region of 12C9. Optionally any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 12C9 are fused to an immunoglobulin constant region of the IgG type, optionally a human constant region, optionally a human IgG1, IgG2, IgG3 or IgG4 isotype, optionally further comprising an amino acid substitution, for example to modify (e.g. reduce) effector function (binding to human Fcy receptors) or to provide for conjugation of a moiety of interest (e.g., a detectable moiety).

In one aspect, the anti-MICA antibody comprises a heavy chain variable region having at least about 80% sequence identity (e.g., at least about 85%, 90%, 95%, 97%, 98%, 99% or more identity) to the heavy chain variable region having the amino acid sequence of SEQ ID NO: 7.

In one aspect, the anti-MICA antibody comprises a light chain variable region having at least about 80% sequence identity (e.g., at least about 85%, 90%, 95%, 97%, 98%, 99% or more identity) to the light chain variable region having the amino acid sequence of SEQ ID NO: 8.

In one aspect, the antibody comprises: a HCDR1 comprising an amino acid sequence: GYYMN (SEQ ID NO: 9), or a sequence of at least 4 contiguous amino acids thereof, optionally wherein one or more of these amino acids may be substituted by a different amino acid; a HCDR2 comprising an amino acid sequence: TINPYYGSSTYNQKFKG (SEQ ID NO: 10), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: VDGDHGYFDY (SEQ ID NO: 11), or a sequence of at least 4, 5 or 6 contiguous amino acids thereof, optionally wherein one or more of these amino acids may be substituted by a different amino acid; a LCDR1 comprising an amino acid sequence: RSSQSLVHSNGNTYLH (SEQ ID NO: 12), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: KVSTRFS (SEQ ID NO: 13) or a sequence of at least 4, 5 or 6 contiguous amino acids thereof, optionally wherein one or more of these amino acids may be substituted by a different amino acid; and/or a LCDR3 region comprising an amino acid sequence: SQSTHVPFT (SEQ ID NO: 14), or a sequence of at least 4, 5, 6, 7 or 8 contiguous amino acids thereof, optionally wherein one or more of these amino acids may be deleted or substituted by a different amino acid.

The specified heavy chain, light chain, variable region, framework and/or CDR sequences may comprise sequence modifications, e.g. a substitution (1, 2, 3, 4, 5, 6, 7, 8 or more sequence modifications). In one embodiment, an amino acid sequence comprises one, two, three or more amino acid substitutions, where the residue substituted is a residue present in a sequence of human origin. In one embodiment the substitution is a conservative modification. A conservative sequence modification refers to an amino acid modification that does not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified amino acid sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the properties set forth herein) using the assays described herein.

In one embodiment, the antibodies of the invention are antibody fragments that retain their binding and/or functional properties. Fragments and derivatives of antibodies of this invention (which are encompassed by the term “antibody” or “antibodies” as used in this application, unless otherwise stated or clearly contradicted by context), preferably a 12C9-like antibody, can be produced by techniques that are known in the art. “Fragments” comprise a portion of the intact antibody, generally the antigen binding site or variable region. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. In one embodiment, an antibody or antibody fragment is derivatized by conjugating or covalently binding the antibody or antibody fragment to a detectable moiety.

### Preparation and Staining of FFPE Samples

The present antibodies have the particular property of being able to efficiently and specifically bind to polypeptides (e.g. MICA and MICB polypeptides) present in fixed tissue or cell samples. Various methods of preparing and using such tissue preparations are well known in the art, and any suitable method or type of preparation can be used. The antibodies are further capable of binding their target antigen in samples in which therapeutic (e.g. function neutralizing) antibodies were present at or prior to fixation.

The FFPE material in a biological sample from an individual is typically a tissue. FFPE tissue is a piece of tissue which is first separated from a specimen animal (e.g., human individual) by dissection or biopsy. Then, this tissue is fixed in order to prevent it from decaying or degenerating and to permit one to examine it clearly under a microscope for histological, pathological or cytological studies. Fixation is the process by which the tissue is immobilized, killed and preserved for the purpose of staining and viewing it under a microscope. Post-fixation processing makes tissue permeable to staining reagents and cross-links its macromolecules so that they are stabilized and locked in position. This fixed tissue is then embedded in the wax to allow it to be cut into thin sections and be stained with hematoxylin and eosin stain. After that, microtoming is done by cutting fine sections to study stain with antibodies under microscope.

It will be appreciated, for example, that the present antibodies can be used with different suitable fixed cell or tissue preparations, and different particular fixation or embedding methods used. For example, while the most common formaldehyde-based fixation procedure involves formalin (e.g., 10%), alternative methods such as paraformaldehyde (PFA), Bouin solution (formalin/picric acid), alcohol, zinc-based solutions (for one example, see, e.g., Lykidis et al., (2007) Nucleic Acids Research, 2007,1-10, the entire disclosure of which is herein incorporated in its entirety), and others (see, e.g., the HOPE method, Pathology Research and Practice, Volume 197, Number 12, December 2001, pp. 823-826(4), the entire disclosure of which is herein incorporated by reference). Similarly, while paraffin is preferred, other materials can be used for embedding as well, e.g., polyester wax, polyethylene glycol based formulas, glycol methacrylates, JB-4 plastics, and others. For review of methods for preparing and using tissue preparations, see, e.g., Gillespie et al., (2002) Am J Pathol. 2002 February; 160(2): 449-457; Fischer et al. CSH Protocols; 2008; Renshaw (2007), Immunohistochemistry: Methods Express Series; Bancroft (2007) Theory and Practice of Histological Techniques; and PCT patent publication no. WO06074392; the entire disclosures of which are herein incorporated by reference).

In one embodiment of the invention, the FFPE tissue is a tumor tissue or tumor-adjacent tissue, for example human tumor tissue. The tumor may be, for example, tumor of the a head and neck squamous cell carcinoma, a lung cancer (e.g. NSCLC), a mesothelioma, a breast cancer, an estrogen positive breast cancer, an estrogen negative breast cancer, a triple negative breast cancer, an ovarian cancer, an endometrial cancer, a prostate cancer or a melanoma.

The antibody (e.g. anti-MICA and/or MICB antibody) is incubated with the FFPE material for detection of MICA and/or MICB polypeptides. The term incubation step involves the contacting of the FFPE material with the antibody of the invention for a distinct period, which depends on the kind of material, antibody and/or antigen. The incubation process also depends on various other parameters, e.g. the sensitivity of detection, which optimization follows routine procedures known to those skilled in the art. Adding chemical solutions and/or applying physical procedures, e.g. impact of heat, can improve the accessibility of the target structures in the sample. Specific incubation products are formed as result of the incubation.

Suitable tests for the detection of formed antibody/antigen complexes are known to those skilled in the art or can be easily designed as a matter of routine. Many different types of assays are known, examples of which are set forth below.

For example, the sample (tissue or cells) to be examined can be obtained by biopsy from a biological fluid, tumor tissue or from a healthy tissue, and sections (e.g., 3 mm thick or less) and fixed using formalin or an equivalent fixation method (see supra). The time of fixation depends on the application, but can range from several hours to 24 or more hours. Following fixation, the tissue is embedded in paraffin (or equivalent material), and very thin sections (e.g., 5 microns) are cut in a microtome and then mounted onto, preferably coated, slides. The slides are then dried, e.g., air dried.

Fixed and embedded tissue sections on slides can be dried and stored indefinitely. For immunohistochemistry, the slides are deparaffinized and then rehydrated. For example, they are subjected to a series of washes with, initially, xylene, and then xylene with ethanol, and then with decreasing percentages of ethanol in water.

Before antibody staining, the tissues can be subjected to an antigen retrieval step, e.g., enzymatic or heat-based, in order to break methane bridges that form during fixation and which can mask epitopes. In a preferred embodiment, a treatment in boiling 10 mM citrate buffer, pH 6, is used.

Once the slides have been rehydrated and antigen retrieval has been ideally performed, they can be incubated with the primary antibody. First, the slides are washed with, e.g., TBS, and then, following a blocking step with, e.g., serum/BSA, the antibody can be applied. The concentration of the antibody will depend on its form (e.g., purified), its affinity, the tissue sample used, but a suitable concentration is, e.g., 1-10 μg/ml. In one embodiment, the concentration used is 10 μg/ml. The time of incubation can vary as well, but an overnight incubation is typically suitable. Following a post-antibody washing step in, e.g., TBS, the slides are then processed for detection of antibody binding.

The detection method used will depend on the antibody, tissue, etc. used, and can for example involve detection of a luminescent or otherwise visible or detectable moiety conjugated to the primary antibody, or through the use of detectable secondary antibodies. Methods of antibody detection are well known in the art and are taught, e.g., in Harlow et al., Antibodies: A Laboratory Manual, Cold Spring Harbor Laboratory Press, 1st edition (Dec. 1, 1988); Fischer et al. CSH Protocols; 2008; Renshaw (2007), Immunohistochemistry: Methods Express Series; Bancroft (2007) Theory and Practice of Histological Techniques; PCT patent publication no. WO06074392; the entire disclosure of each of which is herein incorporated in its entirety.

Many direct or indirect detection methods are known and may be adapted for use. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the protein is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine.

Indirect labels include various enzymes well known in the art, such as horseradish peroxidase (HRP), alkaline phosphatase (AP), β-galactosidase, urease and the like. The covalent linkage of an anti-MICA antibody to an enzyme may be performed by different methods, such as the coupling with glutaraldehyde. Both, the enzyme and the antibody are interlinked with glutaraldehyde via free amino groups, and the by-products of networked enzymes and antibodies are removed. In another method, the enzyme is coupled to the antibody via sugar residues if it is a glycoprotein, such as peroxidase. The enzyme is oxidized by sodium periodate and directly interlinked with amino groups of the antibody. Other enzyme containing carbohydrates can also be coupled to the antibody in this manner. Enzyme coupling may also be performed by interlinking the amino groups of the antibody with free thiol groups of an enzyme, such as β-galactosidase, using a heterobifunctional linker, such as succinimidyl 6-(N-maleimido) hexanoate. The horseradish-peroxidase detection system can be used, for example, with the chromogenic substrate tetramethylbenzidine (TMB), which yields a soluble product in the presence of hydrogen peroxide that is detectable at 450 nm. The alkaline phosphatase detection system can be used with the chromogenic substrate p-nitrophenyl phosphate, for example, which yields a soluble product readily detectable at 405 nm. Similarly, the β-galactosidase detection system can be used with the chromogenic substrate o-nitrophenyl-β-D-galactopyranoxide (ONPG), which yields a soluble product detectable at 410 nm. A urease detection system can be used with a substrate, such as urea-bromocresol purple.

In one embodiment, the binding of the primary antibody is detected by binding a labeled secondary antibody, preferably a secondary antibody covalently linked to an enzyme such as HRP or AP. In a particularly preferred embodiment, the signal generated by binding of the secondary antibody is amplified using any of a number of methods for amplification of antibody detection. For example, the EnVision method can be used, (see, e.g., U.S. Pat. No. 5,543,332 and European Patent no. 594,772; Kämmerer et al., (2001) Journal of Histochemistry and Cytochemistry, Vol. 49, 623-630; Wiedorn et al. (2001) The Journal of Histochemistry & Cytochemistry, Volume 49(9): 1067-1071; the entire disclosures of which are herein incorporated by reference), in which the secondary antibodies are linked to a polymer (e.g., dextran) that is itself linked to many copies of AP or HRP.

In one example, formalin-fixed paraffin embedded blocks are sliced in 5 μm-thick sections and immunostainings performed on a Discovery Ultra or a Benchmark Ultra automaton (Ventana). After pre-treatment with cell conditioning 1, sections are incubated 1 hour at 37° C. with anti-MICA/B primary antibody or mouse IgG1 isotype control at 2 μg/mL (for staining on the Discovery Ultra) or at 6.6 μg/mL (for staining on the Benchmark Ultra). Then, signal amplification using the discovery Amp HQ™ kit or the UltraView™ kit is performed. After revelation with 3,3-diaminobenzidine, counterstaining with hematoxylin and bluing, sections are washed, dehydrated, cleared and coverslipped. Stained sections are finally scanned on a slide scanner (S60 Nanozoomer™, Hamamatsu or a Pannoramic scan II, 3DHistech™). Staining is interpreted and scored by trained pathologists that determine the MICA/B expression on the tumor cells. Samples with more than a specified percentage (e.g. 1%, 5%, 10% etc.) of MICA/B positive tumor cells are considered MICA/B positive.

### Compositions and Uses in Diagnostics, Prognostics and Therapy

The antibodies of the disclosure are particularly effective at detecting MICA and/or MICB (e.g. including a plurality of the most predominant alleles of MICA in the human population, and including at least MICA*001 and MICA*008 alleles) within biological samples prepared as FFPE, without non-specific staining on tissues or cells that do not express MICA or MICB polypeptides. The antibodies will therefore have advantages for use in the study, evaluation, diagnosis, prognosis and/or monitoring in diseases where detection of and/or localization of MICA and/or MICB polypeptide and/or MICA and/or MICB-expressing cells is of interest. For example, patients whose tumor or tumor adjacent tissues are characterized by MICA and/or MICB -expressing cells (e.g. MICA and/or MICB-expressing tumor cells) may have an unfavorable prognosis for tumor progression. Such patients may for example benefit from treatment with therapeutic agents and regimens adapted for their prognosis and/or MICA and/or MICB expression profile, including for example immunotherapies, chemotherapies and particular combination treatments.

Accordingly, provided are methods of detecting, diagnosing, or monitoring cancer in a subject, the method comprising the steps of contacting (e.g. in vitro) tumor cells with an anti-MICA/MICB antibody or antibody fragment of the disclosure and detecting the tumor-associated MICA and/or MICB polypeptide. In related embodiments the diagnostic method will comprise immunohistochemistry (IHC). In certain embodiments, the tumor sample is chemically fixed and/or paraffin embedded. Those of skill in the art will further appreciate that such MICA/MICB detection agents may be labeled or associated with effectors, markers or reporters as and detected using any one of a number of standard imaging techniques. In other embodiments the anti-MICA/MICB antibody will not be directly labelled and will be detected using a secondary agent that is detectable (e.g., a labelled anti-murine antibody). In certain embodiments, the present disclosure provides a method for identifying or selecting an individual for administration of a therapy (e.g. a chemotherapy, an immunotherapy) comprising diagnosing an individual using any of the anti-MICA/MICB compositions and detection methods of the invention, and tailoring a course of therapy based on the outcome.

In one embodiment, the disclosure provides a method of detecting MICA/MICB expressing cells in a sample from an individual having a head and neck squamous cell carcinoma, a lung cancer (e.g. NSCLC), a mesothelioma, a breast cancer, an estrogen positive breast cancer, an estrogen negative breast cancer, a triple negative breast cancer, an ovarian cancer, an endometrial cancer, a prostate cancer or a melanoma, the method comprising bringing a paraffin-embedded tumor tissue sample from the individual into contact with an antibody capable of specifically binding to human MICA and MICB polypeptides in a paraffin-embedded tumor tissue sample; and detecting the presence of bound antibody within the section, optionally further detecting cell membrane (cell surface) staining by the antibody. Upon a detection of bound antibody within the section, optionally a detection of cell membrane (cell surface) staining by the antibody within the section, the individual can be determined or deemed as having a tumor that is MICA and/or MICB positive.

In one embodiment, the disclosure provides a method of detecting MICA/B expressing cells in a sample from an individual having a urothelial cancer, pancreatic cancer, hepatocellular carcinoma (HCC) or endometrial cancer, the method comprising bringing a paraffin-embedded tumor tissue sample from the individual into contact with an antibody capable of specifically binding to human MICA and MICB polypeptides in a paraffin-embedded tumor tissue sample; and detecting the presence of bound antibody within the section, optionally further detecting cell membrane (cell surface) staining by the antibody. Upon a detection of bound antibody within the section, optionally a detection of cell membrane (cell surface) staining by the antibody within the section, the individual can be determined or deemed as having a tumor that is MICA and/or MICB positive.

In one embodiment, the disclosure provides a method of detecting MICA/B expressing cells (e.g. cells expressing MICA and/or MICB at their surface or cell membrane) in a sample from a human tumor, the method comprising bringing a paraffin-embedded tumor tissue sample from the individual into contact with an antibody capable of specifically binding to human MICA and MICB polypeptides in a paraffin-embedded tumor tissue sample; and detecting the presence of bound antibody within the section, optionally further detecting cell membrane (cell surface) staining by the antibody.

In one embodiment, the disclosure provides a method of detecting MICA/B expressing cells in a sample from an individual having a head and neck squamous cell carcinoma, a lung cancer (e.g. NSCLC), a mesothelioma, a breast cancer, an estrogen positive breast cancer, an estrogen negative breast cancer, a triple negative breast cancer, an ovarian cancer, an endometrial cancer, a prostate cancer or a melanoma, the method comprising bringing a paraffin-embedded tumor tissue sample from the individual into contact with an antibody capable of specifically binding to human MICA and MICB polypeptides in a paraffin-embedded tumor tissue sample; and detecting the presence of bound antibody within the section, optionally further detecting cell membrane (cell surface) staining by the antibody.

In one embodiment, the disclosure provides a method of detecting MICA/B expressing cells in a sample from a human tumor, the method comprising bringing a paraffin-embedded tumor tissue sample from the individual into contact with an antibody capable of specifically binding to human MICA and MICB polypeptides on the surface of cells that have been prepared as a paraffin-embedded cell pellet; and detecting the presence of bound antibody within the section, optionally further detecting cell membrane (cell surface) staining by the antibody.

In one embodiment, the disclosure provides a method of detecting MICA/B expressing cells in a sample from a human tumor, the method comprising bringing a paraffin-embedded tumor tissue sample from the individual into contact with an antibody that has been assessed for its ability to specifically bind (or has been determined to specifically bind) to human MICA and MICB polypeptides on the surface of cells that have been prepared as a paraffin-embedded cell pellet; and detecting the presence of bound antibody within the section, optionally further detecting cell membrane (cell surface) staining by the antibody.

In one embodiment, the disclosure provides a method of detecting MICA/B expressing cells in a tumor tissue sample from an individual having a tumor, the method comprising:
(a) providing a paraffin-embedded tumor tissue sample from the individual, and bringing the sample into contact, in vitro, with a monoclonal antibody capable of binding to MICA and/or MICB-expressing cells in a paraffin-embedded cell pellet without binding to MICA/MICB-negative cells in a paraffin-embedded cell pellet, and
(b) assessing whether the antibody binds to the surface of tumor cells, wherein a determination that the antibody binds to the surface of tumor cells indicates that the tumor is positive for MICA/B expressing cells and/or is suitable for treatment with a depleting anti-MICA agent.

In any embodiment, the FFPE tissue may be a tumor or tumor-adjacent tissue obtained from in individual who has received prior treatment with an anti-cancer treatment (e.g., chemotherapeutic agent), optionally an anti-cancer treatment that is known to be capable of upregulating MICA and/or MICB expression cancer cells. It has been shown that certain chemotherapeutic agents or other treatments can induce and/or increase MICA and/or MICB expression (and optionally further NKG2D ligands) on tumor cells. This includes well known chemotherapies including ionizing and UV radiation, inhibitors of DNA replication, inhibitors of DNA polymerase, chromatin modifying treatments, anti-metabolite agents (e.g. halogenated analogs of pyruvate such as 3-bromopyruvate) as well as apoptosis inducing agents such as HDAC inhibitors trichostatin A and valproic acid. Exemplary agents are those that activate the DNA damage response pathway, for example those that activate the ATM (ataxia telangiectasia, mutated) or ATR (ATM- and Rad3-related) protein kinases, or CHK1, or yet further CHK2 or p53. Examples of the latter include ionizing radiation, inhibitors of DNA replication, DNA polymerase inhibitors and chromatic modifying agents or treatment including HDAC inhibitors. Compositions that upregulate NKG2D ligands are further described in Gasser et al (2005) Nature 436(7054): 1186-90. Further chemotherapeutic agents include alkylating agents, cytotoxic antibiotics such as topoisomerase I inhibitors, topoisomerase II inhibitors, plant derivatives, RNA/DNA antimetabolites, and antimitotic agents. Preferred examples may include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, taxol, gemcitabine, navelbine, transplatinum, 5-fluorouracil, vincristin, vinblastin and methotrexate, or any analog or derivative variant of the foregoing. An individual who has received prior treatment with an anti-cancer treatment and who has MICA and/or MICB expression on tumor cells may be determined to be suitable for treatment with an anti-MICA and/or MICB antibody.

In one embodiment, when MICA and/or MICB is detected, the FFPE tissue may be a tumor or tumor-adjacent tissue obtained from in individual who is a candidate for treatment with an anti-MICA and/or MICB antibody.

In one embodiment, when MICA and/or MICB is detected, the FFPE tissue may be a tumor or tumor-adjacent tissue obtained from in individual who has received treatment with an anti-MICA and/or MICB antibody, e.g., who has undergone or is undergoing a course of therapy with such antibody.

The disclosure further provides a method for selecting individuals having a MICA and/or MICB-expressing tumor for administering therapeutic interventions. Examples of therapeutic interventions include an antibody that binds MICA and/or MICB, optionally an antibody that causes the depleting of MICA and/or MICB-expressing cells. In one example, a therapeutic intervention is an agent (e.g. an antibody that binds MICA) that relieves or reduces the immunosuppressive effect of MICA and/or MICB, for example an agent that relieves or reduces the down-modulation of NKG2D at the surface of NK and/or CD8 T cells induced by soluble MICA polypeptides. If the individual is determined or deemed to have a tumor that is MICA and/or MICB positive, the methods of the disclosure can optionally be further be specified as comprising a step of treating the individual with the therapeutic intervention, e.g. if the individual is determined to have a MICA and/or MICB-expressing tumor, as determined by staining in a FFPE sample, optionally cell membrane (cell surface) staining, using an antibody capable of specifically binding human MICA and/or MICB in a FFPE sample.

In any embodiment, the antibody can be used without an additional or prior step of determining or assessing which allele(s) of MICA is expressed by an individual. In any embodiment, the antibody can be used across the human population.

The antibodies described herein can be used for the detection, preferably in vitro, of the presence MICA and/or MICB-expressing cells, for example tumor cells. Such a method will typically involve contacting a biological sample (e.g. a FFPE sample, deparaffinized) from an individual with an antibody according to the disclosure and detecting the formation of immunological complexes resulting from the immunological reaction between the antibody and the biological sample. The complex can be detected directly by labelling the antibody according to the disclosure or indirectly by adding a molecule which reveals the presence of the antibody according to the invention (secondary antibody, streptavidin/biotin tag, etc.). For example, labelling can be accomplished by coupling the antibody with radioactive or fluorescent tags. These methods are well known to those skilled in the art. Accordingly, the invention also relates to the use of an antibody according to the disclosure for preparing a diagnostic composition that can be used for detecting the presence of MICA and/or MICB-expressing cells (e.g., tumor cells), optionally for detecting the presence of a pathology where MICA and/or MICB-expressing cells are present, optionally for characterizing a cancer or other pathology, in vivo or in vitro.

In some embodiments, the antibodies of the disclosure will be useful for predicting cancer progression. A cancer prognosis, a prognostic for cancer or cancer progression comprises providing the forecast or prediction of (prognostic for) any one or more of the following: duration of survival of a subject susceptible to or diagnosed with a cancer, duration of recurrence-free survival, duration of progression free survival of a subject susceptible to or diagnosed with a cancer, response rate to treatment in a subject or group of subjects susceptible to or diagnosed with a cancer, and/or duration of response, degree of response, or survival following treatment in a subject. Exemplary survival endpoints include for example TTP (time to progression), PFS (progression free survival), DOR (duration of response), and OS (overall survival). Generally, disease progression and responses can be determined according to standard tumor response criteria conventions, for example according to “Response Evaluation Criteria in Solid Tumors” (RECIST) v1.1 as detailed by Eisenhauer, E A, et al, New response evaluation criteria in solid tumours: Revised RECIST guideline (version 1.1), Eur J Cancer 2009:45:228-247; the disclosure of which is incorporated by reference herein.

Diagnosing a MICA/MICB positive tumor, predicting cancer progression and/or tailoring a therapeutic regimen to an individual having cancer may for example be based on the measurement of percent of positively stained MICA cells in a tumor or tumor adjacent tissue sample. In this regard patients exhibiting a certain percentage of positively stained cells in a fixed IHC sample when interrogated with an anti-MICA/MICB antibody would be considered MICA/MICB+ and would be selected for treatment in accordance with the teachings herein. In such embodiments tumor samples exhibiting greater than 1%, greater than 5%, greater than 10%, greater than 20%, greater than 30%, greater than 40% or greater than 50% positive cell staining may be classified as MICA/MICB+ when measured as percent positive cells. In certain aspects the MICA/MICB+ tumor will express MICA and/MICB in >50% of the constituent cells when measured as percent positive.

In other embodiments patient diagnosis and/or selection may be predicated on the percent of MICA and/or MICB positive cells staining with a certain intensity. By way of example, a tumor with >10%, optionally >20%, of the cells exhibiting 2+ intensity or greater may be deemed to be suitable for treatment with a chemotherapeutic or immunotherapeutic agent. In other embodiments a patient will be a candidate for treatment with a chemotherapeutic or immunotherapeutic agent if >10%, >20%, >30%, >40% or >50% of the tumor cells exhibit 1+ intensity or greater when stained with an anti-MICA/MICB antibody and examined in accordance with standard IHC protocols, e.g., as disclosed herein. In other certain embodiments an individual will be suitable for treatment with a chemotherapeutic or immunotherapeutic agent if >10%, >20%, >30%, >40% or >50% of the tumor cells exhibit 2+ intensity or greater when stained with an anti-MICA/MICB antibody and examined in accordance with standard IHC protocols, e.g., as disclosed herein.

In some aspects, a tissue sample (e.g. tumor or tumor-adjacent tissue sample) from an individual having a cancer can be characterized or assessed using an antibody disclosed herein to assess MICA and/or MICB polypeptide and/or MICA and/or MICB-expressing cells in the tumor or at the tumor periphery.

In one embodiment, a cancer or tumor characterized by MICA and/or MICB-expressing cells (or an individual having such cancer or tumor) can be identified as being suitable for (e.g. benefitting from) treatment with a chemotherapeutic agent or an immunotherapeutic agent (e.g. a depleting anti-MICA and/or -MICB antibody).

In one embodiment, the disclosure provides an in vitro method for the diagnosis, prognosis, monitoring and/or characterization of a cancer in an individual in need thereof, the method comprising providing a paraffin-embedded tumor or tumor-adjacent sample from an individual, and detecting MICA and/or MICB polypeptide (e.g. MICA and/or MICB-expressing cells) in the sample using a monoclonal antibody that specifically binds to a human MICA and MICB polypeptides in a fixed tissue sample, optionally a paraffin-embedded tissue sample, wherein a detection of MICA and/or MICB polypeptide indicates that the individual is amenable to (e.g. benefitting from) treatment with a chemotherapeutic agent or an anti-MICA and/or MICB therapeutic agent. The anti-MICA and/or MICB therapeutic agent may for example be an agent that bind a human MICA and MICB polypeptide, optionally wherein the agent is a depleting agent, for example an anti-MICA antibody (or function-conservative variant thereof) having the heavy and light chain CDRs or variable regions of any of the known antibodies disclosed in WO2013/117647, WO2013/049527, WO2014/040903, WO2015/085210, WO2018/217688 (e.g., 7C6 antibody), WO2018/081648 and WO2019/183551 (e.g. 1D5 antibody), the disclosures of which are incorporated herein by reference. Such agents may be useful to treat individuals having tumors or tumor tissue characterized by detectable and/or elevated levels MICA and/or MICB expression.

In one embodiment, the disclosure provides a method for the treatment or prevention of a head and neck squamous cell carcinoma, a lung cancer (e.g. NSCLC), a mesothelioma, a breast cancer, an estrogen positive breast cancer, an estrogen negative breast cancer, a triple negative breast cancer, an ovarian cancer, an endometrial cancer, a prostate cancer, a melanoma, a urothelial cancer, a pancreatic cancer, a hepatocellular carcinoma (HCC) or an endometrial cancer in an individual in need thereof, the method comprising:
a) detecting MICA and/or MICB polypeptide, optionally at the surface of cells (e.g., membranar MICA and/or MICB staining) in a formalin-treated and/or paraffin-embedded tumor tissue sample (or tumor cell sample) from the individual, and
b) upon a determination that tumor sample (or tumor cells) comprises MICA polypeptide (e.g. MICA-expressing cells), optionally at a level that is increased compared to a reference level, administering to the individual an anti-cancer agent, optionally an antibody that binds a human MICA and/or MICB polypeptide or a chemotherapeutic agent. In one embodiment, the individual has received prior treatment with a chemotherapeutic agent (prior to step (a)). Detecting MICA and/or MICB polypeptide can be carried using an antibody of the disclosure.

In any aspect, detecting MICA and/or MICB polypeptide in sample using an antibody can comprise the steps of contacting a biological sample (e.g. a FFPE sample, deparaffinized) from an individual with the antibody and detecting the formation of immunological complexes resulting from the immunological reaction between the antibody and the biological sample.

Also provided are diagnostic or prognostic kits, e.g., for cancer, comprising an antibody according to the disclosure for detection of MICA and/or MICB. Optionally the kit comprises an antibody of the invention and an antibody (e.g. 1, 2, 3, 4, 5, 10 or more antibodies) that binds a non-MICA/MICB polypeptide, for use as a diagnostic or prognostic. Said kit can additionally comprise means by which to detect the immunological complex resulting from the immunological reaction between a biological (e.g. tumor tissue) sample and an antibody, in particular reagents enabling the detection of said antibody.

The present methods may be useful in the study, evaluation, diagnosis, prognosis, and/or monitoring of a range of cancers, for example a head and neck squamous cell carcinoma, a lung cancer (e.g. NSCLC), a mesothelioma, a breast cancer, an estrogen positive breast cancer, an estrogen negative breast cancer, a triple negative breast cancer, an ovarian cancer, an endometrial cancer, a prostate cancer, a melanoma, a urothelial cancer, a pancreatic cancer, a hepatocellular carcinoma (HCC) or an endometrial cancer.

### EMBODIMENTS

1. An antibody or antibody fragment capable of specifically binding to a human MICA polypeptide and to a human MICB polypeptide, wherein the antibody or antibody fragment comprises the three CDRs of the heavy chain variable region sequence of SEQ ID NO: 7 and the three CDRs of the light chain variable region sequence of SEQ ID NO: 8, wherein CDRs are determined according to Kabat numbering.
2. An antibody or antibody fragment capable of specifically binding to human MICA and MICB polypeptides, wherein such binding is in a sample of cells that express such MICA and/or MICB polypeptide and that have been prepared as a paraffin-embedded cell pellet, wherein the antibody or antibody fragment comprises a heavy chain variable domain comprising an amino acid sequence at least 80%, optionally at least 90%, identical to the amino acid sequence of SEQ ID NO: 7, and a light chain variable domain comprising an amino acid sequence at least 80%, optionally at least 90%, identical to the amino acid sequence of SEQ ID NO: 8.
3. The antibody or antibody fragment of embodiments 1 or 2, wherein the antibody or antibody fragment is conjugated or covalently bound to a detectable moiety.
4. The antibody or antibody fragment of any one of the above embodiments, wherein the antibody or antibody fragment binds to a MICA polypeptide in a sample of MICA-expressing cells that has been prepared as a paraffin-embedded cell pellet, but does not bind to MICA- and MICB-negative cells, optionally Raji cells, that have been prepared as a paraffin-embedded cell pellet.
5. The antibody or antibody fragment of any one of the above embodiments, wherein the antibody or antibody fragment binds to a MICA*001 polypeptide in a sample of MICA*001-expressing cells that has been prepared as a paraffin-embedded cell pellet, and that further binds to a MICA*008 polypeptide in a sample of MICA*008-expressing cells that has been prepared as a paraffin-embedded cell pellet.
6. The antibody or antibody fragment of any one of the above embodiments, wherein the antibody or antibody fragment binds to a MICB polypeptide in a sample of MICB-expressing cells that has been prepared as a paraffin-embedded cell pellet, but does not bind to MICA- and MICB-negative cells, optionally Raji cells, that have been prepared as a paraffin-embedded cell pellet.
7. The antibody or antibody fragment of any one of the above embodiments, wherein antibody or antibody fragment binds to BxPC-3 cells that have been prepared as a paraffin-embedded cell pellet; optionally wherein the antibody or antibody fragment is capable of staining BxPC-3 cells that have been prepared as a paraffin-embedded cell pellet when the antibody is provided at low concentration (1 μg/mL), optionally further wherein the antibody or antibody fragment is capable of staining BxPC-3 cells that have been prepared as a paraffin-embedded cell pellet, when the antibody is provided at low concentration (1 μg/mL), at medium (5 μg/mL) and at high concentrations (10 μg/mL).
8. The antibody or antibody fragment of any one of the embodiments 2-7, wherein the antibody or antibody fragment comprises the three CDRs of the heavy chain variable region sequence of SEQ ID NO: 7 and the three CDRs of the light chain variable region sequence of SEQ ID NO: 8, wherein CDRs are determined according to Kabat numbering.
9. The antibody or antibody fragment of any one of the above embodiments, wherein the antibody or antibody fragment competes with an antibody comprising a heavy chain variable region having the amino acid sequence of SEQ ID NO: 7 and a light chain variable region having the amino acid sequence of SEQ ID NO: 8, for binding to a human MICA polypeptide expressed by cells (e.g. MICA-expressing cells) prepared as a paraffin embedded cell sample.
10. An in vitro method of detecting a MICA and/or a MICB polypeptide within a sample from a human individual, said method comprising providing a paraffin-embedded sample from the individual, and detecting a MICA polypeptide in said sample using an antibody or antibody fragment of embodiments 1-9.
11. An in vitro method of detecting a MICA and/or a MICB polypeptide within a sample from a human individual, said method comprising providing a paraffin-embedded sample from the individual, and detecting a MICA polypeptide in said sample using an antibody or antibody fragment that binds to a human MICA*001 polypeptide in a sample of MICA*001-expressing cells that has been prepared as a paraffin-embedded cell pellet, that binds to a human MICA*008 polypeptide in a sample of MICA*008-expressing cells that has been prepared as a paraffin-embedded cell pellet, and that binds to a human MICB polypeptide in a sample of MICB-expressing cells that has been prepared as a paraffin-embedded cell pellet, but does not bind to MICA- and MICB-negative cells, optionally Raji cells, that have been prepared as a paraffin-embedded cell pellet.
12. The method of embodiment 11, wherein antibody or antibody fragment binds to BxPC-3 cells that have been prepared as a paraffin-embedded cell pellet; optionally wherein the antibody or antibody fragment is capable of staining BxPC-3 cells that have been prepared as a paraffin-embedded cell pellet when the antibody is provided at low concentration (1 μg/mL), optionally further wherein the antibody or antibody fragment is capable of staining BxPC-3 cells that have been prepared as a paraffin-embedded cell pellet, when the antibody is provided at low concentration (1 μg/mL), at medium (5 μg/mL) and at high concentrations (10 μg/mL).
13. The method of embodiments 11 or 12, wherein antibody or antibody fragment is an antibody or antibody fragment of embodiments 1-9.
14. The method of embodiments 11 or 12, wherein the antibody or antibody fragment is an antibody comprising a heavy chain variable region having the amino acid sequence of SEQ ID NO: 7 and a light chain variable region having the amino acid sequence of SEQ ID NO: 8; an antibody or antibody fragment that competes with an antibody comprising a heavy chain variable region having the amino acid sequence of SEQ ID NO: 7 and a light chain variable region having the amino acid sequence of SEQ ID NO: 8, for binding to a human MICA polypeptide expressed by cells prepared as a paraffin embedded cell sample; or a function-conservative variant of the antibody or antibody fragment comprising a heavy chain variable region having the amino acid sequence of SEQ ID NO: 7 and a light chain variable region having the amino acid sequence of SEQ ID NO: 8.
15. The method of any one of embodiments 10 to 14, wherein the step of detecting a MICA and/or a MICB polypeptide comprises contacting the sample with the antibody or antibody fragment and detecting the formation of immunological complexes resulting from the immunological reaction between the antibody or antibody fragment and the sample.
16. The method of any one of embodiments 10 to 14, wherein the step of detecting a MICA and/or a MICB polypeptide comprises contacting the sample with the antibody or antibody fragment and detecting the formation of immunological complexes at the cell membrane resulting from the immunological reaction between the antibody or antibody fragment and the sample.
17. The method any one of embodiments 10 to 16, wherein the sample is tumor tissue.
18. The method any one of embodiments 10 to 17, wherein the individual has a head and neck squamous cell carcinoma, a lung cancer (e.g. NSCLC), a mesothelioma, a breast cancer, an estrogen positive breast cancer, an estrogen negative breast cancer, a triple negative breast cancer, an ovarian cancer, an endometrial cancer, a prostate cancer, a melanoma, a urothelial cancer, a pancreatic cancer, a hepatocellular carcinoma (HCC) or an endometrial cancer.
19. The method of any one of embodiments 10 to 18, wherein the individual is an individual who has received, is receiving or is a candidate for treatment with an anti-MICA antibody or antibody fragment.
20. The method of any one of embodiments 10 to 19, wherein the individual is an individual who has received a prior treatment with a chemotherapeutic agent.
21. The method or composition of any one of the above embodiments, wherein the paraffin-embedded tissue sample has been fixed, embedded in paraffin, sectioned, deparaffinized, and transferred to a slide.
22. The method or composition of any one of the above embodiments, wherein MICA and/or MICB polypeptide is detected using a secondary antibody that specifically binds to the antibody that binds a MICA and/or MICB polypeptide.
23. An in vitro method of assessing MICA and MICB expression in an individual who has received prior treatment with a chemotherapeutic agent, said method comprising providing a paraffin-embedded tumor or tumor adjacent tissue sample from the individual, and detecting a MICA and/or MICB polypeptide in said sample using an a antibody or antibody fragment that binds to a human MICA*001 polypeptide in a sample of MICA*001-expressing cells that has been prepared as a paraffin-embedded cell pellet, that binds to a human MICA*008 polypeptide in a sample of MICA*008-expressing cells that has been prepared as a paraffin-embedded cell pellet, and that binds to a human MICB polypeptide in a sample of MICB-expressing cells that has been prepared as a paraffin-embedded cell pellet, but does not bind to MICA- and MICB-negative cells that have been prepared as a paraffin-embedded cell pellet, wherein a detection of MICA and/or MICB polypeptide indicates that the individual is suitable for treatment with a therapeutic agent, optionally wherein the therapeutic agent is an antibody that binds to a human MICA and/or MICB polypeptide.
24. An in vitro method of assessing the suitability of an individual having a tumor for treatment with a therapeutic agent, said method comprising providing a paraffin-embedded tumor or tumor adjacent tissue sample from the individual, and detecting a MICA and/or MICB polypeptide in said sample using an antibody or antibody fragment of embodiments 1-9 or the method of any one of embodiments 10-22 wherein a detection of MICA and/or MICB polypeptide indicates that the individual is suitable for treatment with a therapeutic agent, optionally wherein the therapeutic agent is an antibody that binds to a human MICA and/or MICB polypeptide.
25. The method of embodiments 23-24, further comprising a step of administering to the individual the antibody that binds to a human MICA and/or MICB polypeptide.
26. The method of embodiments 10-25, wherein the individual has received prior treatment with radiation or a chemotherapeutic agent known to be capable of causing upregulation of MICA and/or MICB expression by tumor cells.
27. A method of predicting cancer progression in an individual having a cancer, said method comprising providing a paraffin-embedded tumor tissue sample from the individual, and detecting MICA polypeptide in said sample according to the method of any one of embodiments 9-21, or using antibody or antibody fragment of any one of embodiments 1-9.
28. The method of embodiment 27, wherein a detection of MICA polypeptide (or a detection of a greater number of such MICA-expressing cells compared to a reference value) in said sample indicates that the individual has an unfavorable prognosis for cancer progression.
29. The method of any one of embodiments 10-28, wherein detecting cells in said sample using an antibody comprises:
   obtaining a biological sample (e.g. as a biopsy) comprising cells;
   fixing, embedding in paraffin, sectioning, and deparaffinizing the sample, and optionally transferring the sample to a slide;
   bringing said section into contact with said antibody; and
   detecting the presence of bound antibody within the section.
30. A kit comprising the antibody or antibody fragment of any one of embodiments 1-9, optionally wherein the kit further comprises a labeled secondary antibody that specifically recognizes the antibody of any one of embodiments 1-9.
31. A kit comprising the antibody or antibody fragment of any one of embodiments 1-9, and a therapeutic, optionally a depleting and/or neutralizing, anti-MICA antibody.
32. A nucleic acid or set of nucleic acids encoding the antibody or antibody fragment of embodiments 1-9.
33. A hybridoma or recombinant host cell producing the antibody of embodiments 1-9 or comprising a nucleic acid(s) of embodiment 32.
34. A method of producing an antibody that specifically binds to human MICA and MICB polypeptides in paraffin-embedded tissues, said method comprising the steps of:
   a) providing cells that express at their surface a MICA polypeptide, providing cells that express at their surface a MICB polypeptide, and providing cells that express at their surface neither MICA nor MICB polypeptide, and for each of said cells, preparing a separate paraffin embedded cell sample;
   b) providing a plurality of candidate antibodies; and
   c) preparing or selecting antibodies from said plurality that bind to the paraffin embedded cells of step a) that express MICA and to the paraffin embedded cells of step a) that express MICB, without binding to the paraffin embedded cells of step a) that express at their surface neither MICA nor MICB.
35. The method of embodiment 34, further comprising the step of making derivatives of the selected antibodies.
36. The method of embodiment 35, wherein making derivatives comprises conjugating or covalently binding the antibody to a detectable moiety.
37. The method of embodiments 34-36, wherein the MICA polypeptide is a MICA*001 polypeptide.
38. The method of embodiments 34-37, wherein the method comprises:
   a) providing cells that express at their surface a MICA*001 polypeptide, providing cells that express at their surface a MICA*008 polypeptide, providing cells that express at their surface a MICB polypeptide, and providing cells that express at their surface neither MICA nor MICB polypeptide, and for each of said cells, preparing a separate paraffin embedded cell sample;
   b) providing a candidate antibody or a plurality of candidate antibodies; and
   c) testing the candidate antibody for binding to, or preparing or selecting antibodies from said plurality of candidate antibodies that bind to, the paraffin embedded cells of step a) that express MICA*001, to the paraffin embedded cells of step a) that express MICA*008 and to the paraffin embedded cells of step a) that express MICB, without binding to the paraffin embedded cells of step a) that express at their surface neither MICA nor MICB.
39. An antibody obtained or produced according to the methods of embodiments 34-38.
40. An antibody obtained or produced according to the methods of embodiments 34-39, for use in a method of detecting a MICA and/or a MICB polypeptide within a sample from a human individual, optionally for use in a method of any one of embodiments 10-29.

Further aspects and advantages of this invention will be disclosed in the following experimental section, which should be regarded as illustrative and not limiting the scope of this application.

### EXAMPLES

### Example 1: Performance of BAMO1 Antibody in FFPE Samples

MICA and its close relative MICB are highly polymorphic ligands of the NK cell activating receptor NKG2D. MICA and MICB are induced at the cell surface by cellular stress such as infections and tumor transformation. Indeed, MICA is specifically expressed on several highly prevalent solid tumors including breast, colorectal and lung. In order to identify therapeutic indications for the anti-MICA/B therapeutic antibody, there was a need to assess the expression of MICA/B in different FFPE tumor samples by immunohistochemistry (IHC). The percentage of positive cells in tumors as well as the level of cytoplasmic or membrane expression will help identify suitable indications for the anti-MICA/B therapeutic antibody.

The aim of this study was to test different approaches to try to improve the sensitivity of MICA/B manual immunostaining using available antibodies. Antibody BAMO1 (R&D Systems Inc.) is described as being able to detect MICA in formalin-fixed paraffin-embedded tissue sections of human pancreas, and was selected for testing.

In order to assess the improvement of MICA/B staining sensitivity, cell lines with a low expression level of MICA/B were selected. The challenge was indeed to detect even low expression by IHC. To assess the antibody for MICA/B detection in FFPE sections, MICA/B positive and negative cells were thawed and cultivated, and then fixed and embedded in paraffin for IHC staining. Table 3 summarizes the cells used.

TABLE 3

| | | Culture | Complete |
|---|---|---|---|
| | Cell lines | properties | growth medium |
| MICA/B positive cells | BxPC-3 | Adherent | RPMI |
| | Hs700T | Adherent | DMEM |
| | HT-29 | Adherent | MacCoy's 5A |
| | MIA PaCa2 | Adherent | DMEM |
| MICA/B negative cells | Raji | Suspension | RPMI |

Previous flow cytometry phenotyping studies allowed us to pre-select different cell lines with a low expression level of MICA/B. To confirm the selection, MICA/B expression level on these cells was assessed again by flow cytometry. BxPC-3, Hs 700T, HT-29, and MIA PaCa-2 cells all expressed MICA/B at a relatively low level.

Once the cell phenotype (MICA/B positive) confirmed by flow cytometry, the cells were fixed and embedded in paraffin for IHC staining.

The results showed that MICA/B expression was clearly detected on MIA PaCa-2 cells using the BAMO1 antibody. On Hs 700T and HT-29 cells, the staining was positive but not on all cells. MICA/B expression was not detected on BxPC-3 cells using the BAMO1 antibody. Despite a MICA/B expression detected by flow cytometry, some cell lines did not show any MICA/B staining by IHC. This suggested that the initial protocol was not sensitive enough to detect low expression level of MICA/B. These cells were thus selected to test the improvement of MICA/B staining sensitivity.

Several conditions were tested to optimize the MICA/B IHC staining and the following conditions were tested:
Wash step: flux vs. bath
Blocking of endogenous peroxidases before vs. after primary incubation
Diaminobenzidine (DAB) incubation: 5 minutes vs. 20 minutes
Tyramide system amplification (TSA)
Envision FLEX kit (linker between primary and secondary-HRP antibodies)

Using TSA, MICA/B expression was detected on MIA PaCa-2 cells, on BxPC-3 cells and to a lesser extent on HT-29 cells. Unfortunately, these results were not reproducible (especially for BxPC3 cells for which staining was inconsistent over repeated experiments) and staining intensity remained low.

The QIFIKIT was used to obtain a quantitative determination of MICA/B cell surface expression. Four experiments were performed with the Qifikit on the cell lines selected. The number of antigens found on the cell surface of the different cell lines was equivalent in the different experiments. The data shown in Table 2 are representative of the 4 experiments. The selected cells can be classified on the basis of cell surface antigenic sites: Mia PaCa-2>BxPC3>Hs700T>HT-29. Of note, the QIFIKIT assesses only the cell surface antigens and not cytoplasmic antigens. Since BAMO1 was unable to detect BxPC3 consistently by IHC, we can hypothesize that Hs700T cells were detected by IHC using antibody BAMO1 because they probably express more cytoplasmic MICA/B than BxPC-3.

TABLE 2

| | Qifikit-ABC value |
|---|---|
| | Calculate |
| Beads-negative control | 1197 |
| HT-29 cells | 6411 |
| Hs 700T cells | 13972 |
| BXPC3 cells | 16319 |
| MIA PACA2 cells | 23062 |

The MICA/B IHC staining using BAMO1 on formalin-fixed paraffin-embedded samples could not be optimized. In addition, since this antibody does not allow to consistently stain cells with low number of cell surface MICA/B proteins, it was considered as problematic for the detection of MICA/B expression by IHC on FFPE samples.

### Example 2: Identification of Antibodies That Stain Cells With Low Cell Surface MICA/B in FFPE Samples

Since no satisfactory antibody for IHC on FFPE samples was available, mice were immunized and screening was performed to identify an antibody that can consistently and specifically stain cells with low MICA/B expression.

Briefly, the immunization was performed using five 5 Balb/c mice immunized with 3 proteins (MICA*001, MICA*008 and MICB). Sera from the five different animals were tested by IHC on C1Rneo, C1R MICA*008 and C1R MICB FFPE cells pellets using. Three animals were selected for the fusion because sera from those animals allowed staining high number of MICA/B cells with strong staining intensities. After hybridoma culture and selection, 508 supernatants (undiluted) were tested by IHC using a manual protocol with 2 antigen retrieval conditions (pH6 and 8), on FFPE cell pellets (mix of C1R MICA*008+C1R MICB cells) and an automated protocol, using the Ventana Discovery Ultra automaton and CC1 or CC2 pretreatment, on Raji, BxPC-3, C1R MICA*001, C1R MICA*008 and C1R MICB FFPE cell pellets. The experiment is further detailed below.

The different cell lines were thawed and sub-cultured. Raji, Cl R-neo, BxPC-3, C1R MICA*001, C1R MICA*008 and C1R MICB were cultured in Roswell Park Memorial Institute medium (RPMI) (Gibco) complemented with 10% decomplemented fetal bovine serum (FBS), 1% L-Glutamine, 1% non-essential amino acid and 1% sodium pyruvate. Raji, C1R-neo, C1R MICA*001, C1R MICA*008 and C1RMICB were grown in suspension. BxPC-3 are adherent cells and were detached using PBS-EDTA 2 mM. C1R MICA*001, C1R MICA*008 and C1RMICB were selected with geneticin 1.8 mg/ml. At the end of the culture and after 6 passages for the Raji cells, 7 or 8 passages for the BXPC3, 2 passages for the C1R-neo, 2 passages for the C1R MICA*001, 3 or passages for the C1R MICA*008 and 3 or 4 passages for the C1RMICB, cells were fixed in formalin and embedded in paraffin.

Before embedding, cells were stained with anti-MICA/B monoclonal Ab (mAb; clone 19E9, see WO2013/117647) (PE-conjugated), anti-MICA monoclonal Ab (mAb; clone 20C6, see WO2013/117647) (PE-conjugated) and commercial anti-MICB monoclonal (mAb; 236511 R&D) (PE-conjugated), and analyzed by flow cytometry to assess MICA/B, MICA and MICB expression. No MICA/B expression was observed on Raji cells and a very low endogenous MICA/B expression on C1R-neo. Low MICA expression was observed on BxPC-3 cells. Finally, C1R MICA*001 and C1R MICA*008 cells were found strongly MICA positive and C1R MICB cells MICB positive.

The characteristics of the cell lines were used in this study are summarized as follows:
Raji cells (organism: Homo sapiens, human/cell type: B lymphocyte/tissue: lymphoblast/disease: Burkitt's lymphoma/origin: ATCC) with no MICA/B expression
Cl R-neo cells (organism: Homo sapiens, human/cell type: B lymphoblast; Epstein-Barr virus transformed/tissue: peripheral blood lymphoma/ATCC ref. CRL-2369) with very low endogenous expression of MICA/B
BxPC-3 cells (organism: Homo sapiens, human/tissue: pancreas/disease: adenocarcinoma lymphoma/ATCC ref. CRL-1687) with low endogenous MICA expression
C1R MICA*001 cells: C1R-neo cells transfected with human MICA*001 (high MICA*001 expression level)
C1R MICA*008 cells: C1R-neo cells transfected with human MICA*008 (high MICA*008 expression level)
C1R MICB: C1R-neo cells transfected with human MICB*002 (high MICB expression level)

In parallel to the phenotyping by flow cytometry, the cell lines were fixed in formalin and embedded in paraffin. Briefly, 20×10⁶ to 40×10⁶ cells were fixed for 1 hour with formalin 4%. The cells were washed twice in PBS, and there were resuspended in Histogel. The cell pellets were dehydrated and embedded in paraffin. For each cell line, several FFPE cell pellets were made A mix of cells (C1R MICA*008 (15.10⁶ cells) and C1R MICB (15.10⁶ cells)) was also were embedded in paraffin. Sectioning of FFPE cell pellets was performed and MICA/B staining done by IHC. Briefly, 5 μm-thick sections were incubated dewaxed, submitted to an antigen retrieval step and incubated with immune or non-immune sera, hybridoma supernatants, chain combinations Abs or purified and commercial Abs followed by a signal amplification step. Enzymatic revelation was finally performed using 3, 3′-diaminobenzidine (DAB). For the IHC stainings with the mouse sera, staining interpretation was done by attributing a percentage of stained cells in the pellet section as well as an intensity score using the following criterion: “−”: negative staining; “+”: weak positive staining; “++”: intermediate IHC signal in intensity and “+++”: strong positive staining.

Five Balb/c mice were first immunized using a mix of MICA/B recombinant proteins (MICA*001, MICA*008 and MICB) (2 intraperitoneal injections) and sera tested by IHC. Pre-immune and immune sera were tested by IHC on C1R neo cells, and mix of C1R MICA*008+C1R MICB FFPE cell pellets at 3 different dilutions (1/1000, 1/5000 and 1/10000) and 3 different antigen retrieval conditions (pH6, pH8 and pH9).

No staining was seen using the pre-immune sera. Since weak and heterogeneous stainings were obtained on mix of C1R MICA*008+C1R MICB when sera were diluted at 1/5000 or 1/10000, it was decided to perform a third intraperitoneal injection to enhance immune response. Sera from the 5 mice were tested again by IHC after this third injection and we observed an overall stronger reactivity after the third injection. Sera from 3 mice gave best results (higher numbers of C1R MICA*008 and C1R MICB stained cells with strong staining intensities). In comparison, sera from another mouse stained C1R MICA*008 and C1R MICB cells with a lower intensity; and another mouse stained fewer numbers of C1R MICA*008 cells when used at 1/5000 and 1/10000 and obtained no staining C1R MICB cells when used at 1/5000 and 1/10000. The 3 mice that gave best results were chosen for the final boost (intravenous injection of mix MICA/B recombinant protein). Animals were euthanized and their spleen removed and used as a source of cells for fusion with myeloma cells. After culture in methylcellulose semi-solid medium, hybridoma colonies were picked and cultured in 27 different 96-well plates. Then, an ELISA was performed to select only the mouse IgG-secreting hybridomas. This test was followed by another ELISA and the positive hybridomas (IgG secreting) previously identified were tested to eliminate anti-tag hybridomas. At the end, 508 hybridomas were kept, amplified and 1.5 mL of supernatant/hybridoma produced.

The supernatants were tested first by IHC on mix of C1R MICA*008+C1R MICB FFPE cell pellet sections using different antigen unmasking conditions (pH6, pH8). Among the 508 supernatants, 46 (among them 11 positive to both pH6 and pH8) were selected as giving the best results on mix of C1R MICA*008+C1R MICB cells (strong positive and homogenous staining).

The 46 supernatants, that gave staining on mix of C1R MICA*008+C1R MICB cells were tested again by IHC on C1R-neo, BxPC-3, CR1 MICA*001, C1R MICA*008 and C1R MICB FFPE cell pellet sections on Ventana with CC1 or CC2 pre-treatments. Because they gave the strongest stainings on all positives cells tested and no or weak staining on C1R-neo cells, the supernatants 6 supernatants (including 12C9) were selected and produced as a mouse antibody with mouse γ1 (gamma1) chain (mIgG1 isotype).

In parallel 66 supernatants, partially positive on a mix of C1R MICA*008+C1R MICB cells were again tested on Raji, BxPC-3, CR1 MICA*001, C1R MICA*008, C1R MICB FFPE cell pellet sections on Ventana. Because they gave the strongest stainings and were specific for MICA or MICB, three supernatants were selected and produced (one that was MICA specific and two that were MICB specific).

After transitional transfection, different rearrangements were obtained for each antibody selected and tested by IHC on Raji, BxPC-3, C1R MICA*001, C1R MICA*008, C1R MICB FFPE cell pellet sections on the Ventana Discovery Ultra automaton using CC1 Discovery Cell Conditioning 1 (CC1) or RiboCC (CC2) pre-treatment conditions. Six antibodies gave positive staining on all the cells tested and no staining on Raji cells. These antibodies were selected and produced as a mouse antibody with mouse γ1 (gamma1) chain (mIgG1 isotype).

The produced and purified antibodies were tested by IHC in the Raji, BxPC-3, C1R MICA*001, C1R MICA*008, C1R MICB FFPE cell pellet sections at 3 different concentrations (1, 5 and 10 μg/mL) on the Ventana automaton using CC1 or CC2 pre-treatment conditions.

Three antibodies tested (including 12C9) gave strong membranous staining on MICA and MICB transfected cells and no staining on Raji cells. With conditions of staining tested, two of the antibodies showed no staining or very weak staining on BxPC-3 at low concentration (1 μg/mL) and heterogeneous staining at medium (5 μg/mL) and high concentrations (10 μg/mL). Only 12C9 displayed the ability to stain BxPC-3 FFPE cell pellet sections at the three concentrations tested.

The amino acid sequences of the heavy and light chain variable regions of 12C9 are shown below (Kabat CDRs underlined).

12C9 heavy chain variable region (VH):
(SEQ ID NO: 7)
EIQLQQSGPELEKPGASVKISCKASGYAFTGYYMNWMKQRNGKSLDWIGT
INPYYGSSTYNQKFKGKATLTVDESSSTAYMQLTSLTSEDSAVYYCARVD
GDHGYFDYWGRGTTLTVSS
12C9 light chain variable region (VL):
(SEQ ID NO: 8)
DWMTQTPLSLPVSLGDQASISCRSSQSLVHSNGNTYLHWYLQKPGQSPKL
LIYKVSTRFSGVPDRFSGSGSGTDFTLKISRVEAEDLGVYFCSQSTHVPF
TFGSGTKLEIK

1-27. (canceled)

28. An antibody or antibody fragment capable of specifically binding to a human major histocompatibility complex class I-related chain A (MICA) polypeptide and to a human (major histocompatibility complex class I-related chain B (MICB) polypeptide, wherein the antibody or antibody fragment comprises the three complementarity-determining regions (CDRs) of the heavy chain variable region sequence of SEQ ID NO: 7 and the three CDRs of the light chain variable region sequence of SEQ ID NO: 8.

29. The antibody or antibody fragment of claim 28, wherein the antibody or antibody fragment is conjugated or covalently bound to a detectable moiety.

30. The antibody or antibody fragment of claim 28, wherein antibody or antibody fragment binds to BxPC-3 cells that have been prepared as a paraffin-embedded cell pellet; optionally wherein the antibody or antibody fragment is capable of staining BxPC-3 cells that have been prepared as a paraffin-embedded cell pellet when the antibody is provided at low concentration (1 μg/mL), optionally further wherein the antibody or antibody fragment is capable of staining BxPC-3 cells that have been prepared as a paraffin-embedded cell pellet, when the antibody is provided at low concentration (1 μg/mL), at medium concentration (5 μg/mL) and at high concentrations (10 μg/mL).

31. An in vitro method of detecting a MICA and/or a MICB polypeptide within a sample from a human individual, said method comprising providing a paraffin-embedded sample from the individual, and detecting a MICA polypeptide in said sample using an antibody or antibody fragment of claim 28.

32. The method of claim 31, wherein the paraffin-embedded tissue sample has been fixed, embedded in paraffin, sectioned, deparaffinized, and transferred to a slide.

33. The method of claim 31, wherein MICA and/or MICB polypeptide is detected using a secondary antibody that specifically binds to the antibody that binds a MICA and/or MICB polypeptide.

34. An in vitro method of detecting a MICA and/or a MICB polypeptide within a sample from a human individual, said method comprising providing a paraffin-embedded sample from the individual, and detecting a MICA polypeptide in said sample using an antibody or antibody fragment that binds to a human MICA*001 polypeptide in a sample of MICA*001-expressing cells that has been prepared as a paraffin-embedded cell pellet, that binds to a human MICA*008 polypeptide in a sample of MICA*008-expressing cells that has been prepared as a paraffin-embedded cell pellet, and that binds to a human MICB polypeptide in a sample of MICB-expressing cells that has been prepared as a paraffin-embedded cell pellet, but does not bind to MICA- and MICB-negative cells, optionally Raji cells, that have been prepared as a paraffin-embedded cell pellet.

35. The method of claim 34, wherein antibody or antibody fragment binds to BxPC-3 cells that have been prepared as a paraffin-embedded cell pellet; optionally wherein the antibody or antibody fragment is capable of staining BxPC-3 cells that have been prepared as a paraffin-embedded cell pellet when the antibody is provided at low concentration (1 μg/mL), optionally further wherein the antibody or antibody fragment is capable of staining BxPC-3 cells that have been prepared as a paraffin-embedded cell pellet, when the antibody is provided at low concentration (1 μg/mL), at medium (5 μg/mL) and at high concentrations (10 μg/mL).

36. The method of claim 34, wherein the step of detecting a MICA and/or a MICB polypeptides comprises contacting the sample with the antibody or antibody fragment and detecting the formation of immunological complexes resulting from the immunological reaction between the antibody or antibody fragment and the sample.

37. The method of claim 34, wherein the sample is tumor tissue.

38. The method of claim 34, wherein the individual has a head and neck squamous cell carcinoma, a lung cancer, a mesothelioma, a breast cancer, an estrogen positive breast cancer, an estrogen negative breast cancer, a triple negative breast cancer, an ovarian cancer, an endometrial cancer, a prostate cancer, a melanoma, a urothelial cancer, a pancreatic cancer, a hepatocellular carcinoma (HCC) or an endometrial cancer.

39. The method of claim 38, wherein the individual is an individual who has received, is receiving or is a candidate for treatment with an anti-MICA antibody or antibody fragment.

40. The method of claim 39, wherein the individual is an individual who has received a prior treatment with a chemotherapeutic agent.

41. A method of predicting cancer progression in an individual having a cancer, said method comprising providing a paraffin-embedded tumor tissue sample from the individual, and detecting MICA polypeptide in said sample using the antibody or antibody fragment of claim 28.

42. The method of claim 41, wherein detecting cells in said sample comprises:
obtaining a biological sample comprising cells;
fixing, embedding in paraffin, sectioning, and deparaffinizing the sample, and optionally transferring the sample to a slide;
bringing said section into contact with said antibody; and
detecting the presence of bound antibody within the section.

43. A kit comprising the antibody or antibody fragment of claim 28, optionally wherein the kit further comprises a labeled secondary antibody that specifically recognizes said antibody or antibody fragment.

44. A nucleic acid or set of nucleic acids encoding the antibody or antibody fragment of claim 28.

45. A hybridoma or recombinant host cell producing the antibody of claim 28.

46. A method of producing an antibody that specifically binds to human MICA and MICB polypeptides in paraffin-embedded tissues, said method comprising the steps of:
a) providing isolated cells that are made to express at their surface a MICA polypeptide, providing isolated cells that are made to express at their surface a MICB polypeptide, and providing cells that express at their surface neither MICA nor MICB polypeptide, and for each of said cells, preparing a separate paraffin embedded cell sample;
b) providing a plurality of candidate antibodies; and
c) preparing or selecting antibodies from said plurality that bind to the paraffin embedded isolated cells of step a) that express MICA and to the paraffin embedded isolated cells of step a) that express MICB, without binding to the paraffin embedded cells of step a) that express at their surface neither MICA nor MICB.
